(19) United States
(12) Patent Application Publication
(10) Pub. No.: US 2018/0334697 A1
(43) Pub. Date: Nov. 22, 2018

(54) METHOD FOR ISOTHERMAL DNA DETECTION USING A MODIFIED CRISPR/CAS SYSTEM AND THE APPARATUS FOR DETECTION BY SURFACE ACOUSTIC WAVES FOR GENE EDITING

(71) Applicant: Sensor Kinesis Corporation, Los Angeles, CA (US)

(72) Inventors: Yehoshua Shachar, Santa Monica, CA (US); Roger Kornberg, Atherton, CA (US); Ralph Eugene Davis, Mountain View, CA (US)

(73) Assignee: Sensor Kinesis Corporation, Los Angeles, CA (US)

(21) Appl. No.: 15/597,090

(22) Filed: May 16, 2017

(51) Int. Cl. C12P 19/34; A61L 29/16; C12Q 1/68; G01N 27/27

(52) U.S. Cl. CPC C12P 19/34; A61L 29/16; A61K 9/00; G01N 27/27; C12Q 1/68

(57) ABSTRACT

A method of reducing the limit of detection in a surface acoustic wave sensor (SAW) includes the steps of: attaching a plurality of DNA segments to a detection surface of a SAW; performing a CRISPR/Cas9 preparation of the DNA segments to cut and splice a selected protein into at least one of a plurality of the DNA segments; conjugating a nanoparticle to the selected protein; and measuring the number of DNA segments with conjugated nanoparticles using a surface acoustic wave sensor (SAW). The nanoparticle may be modified to form a single electron transistor (SET) which generates a detectable signal in response to RF or ultrasonic excitation which is indicative of binding of the corresponding nanoparticle to a selected target analyte.

### BACKGROUND

### Field of the Technology

The invention relates to the field of surface acoustic wave detectors and an apparatus and method for improving the limit of detection (LOD), while employing an isothermal method for DNA manipulation when modifying a gene sequence, and for a system for delivering such compositions. More specifically, the disclosure relates to modifying a gene sequence using a CRISPR-Cas9 or other nucleic acid editing system and the ability to minimize the LOD with a secondary mass payload attached to the specific gene sequence.

### Description of the Prior Art

The discovery of a prokaryotic viral defense mechanism, clustered regularly interspaced short palindromic repeats (CRISPR-Cas9), has ushered in the ability to target virtually any DNA sequence for binding and cleavage. Essentially the simplest model CRISPR-Cas9 system is composed of two components: a guide RNA 97 nucleotides and a Cas9 protein. The guide RNA 97 nucleotides is the CRISPR part. Clustered regularly interspaced short palindromic repeats (CRISPR) are segments of prokaryotic DNA containing short, repetitive base sequences. Each repetition is followed by short segments of spacer DNA from previous exposures to foreign DNA (e.g., a virus or plasmid). Small clusters of Cas (CRISPR-associated system) genes are located next to CRISPR sequences.

The CRISPR/Cas system is a prokaryotic immune system that confers resistance to foreign genetic elements such as those present within plasmids and phages that provides a form of acquired immunity. RNA harboring the spacer sequence helps Cas proteins recognize and cut exogenous DNA. Other RNA-guided Cas proteins cut foreign RNA. CRISPRs are found in approximately 40% of sequenced bacterial genomes and 90% of sequenced archaea.

A simple version of the CRISPR/Cas system, CRISPR/Cas9, has been modified to edit genomes. By delivering the Cas9 nuclease complexed with a synthetic guide RNA (gRNA) into a cell, the genome can be cut at a desired location, allowing existing genes to be removed and/or new ones added. The last twenty nucleotides at the 3′ end of the RNA is programmed to be homologous to the target DNA sequence. The protein, Cas9, binds the guide RNA which targets a twenty nucleotide DNA sequence. Upon binding to the target, the DNA is cleaved on both strands by two distinct Nickase active sites.

An increasing number of CRISPR-Cas9 type systems from different bacteria species continue to be uncovered. The characterized variations in cleavage sites and subtle differences in mechanism are providing an expanding palette of gene editing tools. Cas9 mutants, which are unable to carry out the DNA cleavage function, create a CRISPR-Cas-DNA complex, which is virtually irreversible under physiological pH and salt conditions. These mutants have been used to fluorescently label sequence specific loci in situ. Gene editing and delivery of Cas9 mRNA by a lipid nanoparticle is generally disclosed in WO2015/191693 incorporated herein by reference.

Surface acoustic wave sensors (SAW) are a class of microelectromechanical systems (MEMS) which rely on the modulation of surface acoustic waves to sense a physical phenomenon. The sensor transduces an input electrical signal into a mechanical wave which, unlike an electrical signal, can be easily influenced by physical phenomena. The device then transduces this wave back into an electrical signal. Changes in amplitude, phase, frequency, or time-delay between the input and output electrical signals can be used to measure the presence of the desired phenomenon. One example of usable SAW device for is described in U.S. Pat. No. 8,436,509 incorporated herein by reference.

What is needed is a way to improve the limit of detection (LOD) of a SAW device and solve the required minimal mass loading associated with the minimum mass' threshold necessary in obtaining a measurable signal, relative to the frequency domain and its time delay, to capture a biological event(s) above the noise floor of the system at its SNR lowest resolution.

### SUMMARY

A high affinity, sequence specific DNA is used in the illustrated embodiments of the invention in a surface acoustic wave (SAW) sensor. The CRISPR/Cas9 methodology of cutting and splicing DNA allows DNA to be bound orthogonally to the detection surface of the SAW device and a protein spliced into the DNA with a nanoparticle conjugated to the protein to enhance its mass, thereby rendering the enhanced mass DNA more readily detectable by the SAW. The limit of detection (LOD) of the SAW is thus substantially decreased, thereby enabling the system to improve the LOD by minimizing the amount of analyte in question needed for detection and to improve resolution. The enhanced SAW device can be used as a counter for conjugation of a specific DNA segment as enabled by the use of the CRISPR methodology.

Gene Editing Using Isothermal DNA Method

In one embodiment, the employment of the CRISPR/Cas9 methodology of cutting, splicing and replicating a DNA segment of interest and altering the DNA structure, is supplemented by exploiting the acousto-electric properties of a gold nanoparticle (GNP) coupled with the guided RNA (gRNA) in the operation of the SAW. The SAW sensor with its shear horizontal wave propagation (at ˜400 MHz) interacts with a conjugated gRNA whereby the functionalized or chemically modified gold nanoparticle operates in a manner suitable for a single electron transistor topology. The functionalized treated GNP acts as an antenna with the characteristics of a quantized acousto-electric current-generating element.

In the SAW-driven pumps the electrons are transported by the SAW along a one-dimensional semiconductor channel by the acousto-electric effect. The acousto-electric effect is a nonlinear phenomenon generating an electric current in a piezo-electric semiconductor and/or in an island insulator (with low capacitive value) formed on a semiconductive layer, where a propagating acoustic wave induces the emission of a single electron. The generated electric current is proportional to the intensity of the acoustic wave and to the value of its electron-induced attenuation. The effect noted above was theoretically predicted in 1953 by R. H. Parmenter, “The acousto-electric effect”, Physical Review, vol. 89, pp. 990-998 (1953) and Its first experimental observation was reported in 1957 by G. Weinreich and H. G. White, “Observation of the acoustoelectric effect”, Physical Review, vol. 106, pp. 1104-1106 (1957).

GNP as Single Electron Transistor (SET)

In one embodiment, where the GNP is formed as a single-electron transistor gate, an attenuation is observed by the effect of the shear acoustic wave propagation, due to a Coulomb blockade (CB) generated with the formation of a small gap insulator-island between two metal electrodes on a gold nanoparticle, simulating a drain-source topology where electron tunneling through a barrier is emitted by the SAW. By employing the semiconductive characteristics of different coatings disposed on the nanoparticle, such as a GNP, we employ a method of activating advanced functions of a transistor gate to enable the emission of a single electron. In the illustrated embodiment the electrical characteristics of a doped SET are used, such as conductance switching, sensitivity to the acousto-electric generating events (“Coulomb blockade staircase” state), whereby the I-V characteristics for the highly asymmetric junction circuit formed across the GNP cause a single electron to move across junctions (capacitive gap between source and drain on the GNP surface) in response to the surface acoustic wave thereby generating the Coulomb blockade staircase response.

In one embodiment, the GNP's are chemically modified (as defined and shown for example by Khondaker S et al, “The fabrication of single-electron transistors using dielectrophoretic trapping of individual gold nanoparticles”) to function as beacons and where such passive device (GNP) element(s) respond to the emitted surface acoustic wave and by exhibiting an electron tunneling phenomenon thereby switching momentarily to emit an electron due to SAW energy propagation, thereby acting as an SET, and where the electronic detector “listens” to the emitted SET event by recording its presence as evidenced by the Coulomb blockade staircase characteristic of the I-V curve.

This application demonstrates the use of GNP with other amperometric value(s) such as conductivity and ohmic parameters can be employed while using GNP nanoparticles as beacons. Using acousto-electric energy due to SAW propagation's wave momentarily attenuates a newly formed junction a single electron transistor (SET), and a suitable listening detector identifies that their presence within the analyte-conjugated assay (to the antibody or the guided RNA), are present. The electrical response of such events formed out of chemically modified gold nanoparticle provides a method for measuring the GNP's presence and for means of counting and recording their presence.

In one other embodiment, we teach a method where nanoparticles are integrated with the gate dielectric of an SET coated by employing SiO₂ through plasma-enhanced chemical vapor deposition (PCVD) to form the simulated source-drain transistor gate to produce momentary acousto-electric effects, or an antenna signature response due to the tunneling effect of an SET in a SAW sensor in response to an acousto-electric event.

The GNP formed as a transistor is fabricated on a heavily doped n-type silicon (n+Si) substrate with a thermally grown oxide layer of up to 100 nm thick. N+Si serves as the gate electrode while the oxide layer functions as the gate dielectric. An example of such fabrication method is reported by Zhegchun Liu et al. IEEE Transactions on Nanotechnology (Volume: 5, Issue: 4, July 2006 Page(s): 379-384).

Variations on the technology of single electron transistor-gate (SET), formed by doping the GNP and its coated surface (SiO₂) are described where the GNP conductive surface is provided with source/drain electrodes which GNP's are also modified by functionalization with a chemical and/or biochemical probe, where switching of the SET in response to an acousto-electric event generated by the SAW is indicative of binding of a target analyte with its matching epitope, where at least some of the impedimetric characteristic of the SET is a response to the SAW acousto-electric event as derviced from the corresponding electrical output change as an output signal, thereby switching the dielectric gate, and providing a measurable unit(s) to a suitable detector.

In one embodiment, the GNP's are chemically modified with linker molecules such as guided RNA (gRNA) may be an organic mono-molecule and the channel region may include a linker layer composed of a self-assembled monomolecular layer formed by a plurality of organic mono-molecules bonded to the substrate of the GNP. The channel region may further include a linker layer composed of a silane compound layer formed on the substrate and having a functional group selected from among an amine group (—NH₂), a carboxyl group (—COOH) and a thiol group (—SH). The functional group selected from among the amine group (—NH₂), carboxyl group (—COOH) and thiol group (—SH) may be a portion(s) of the linkers.

The drawback associated with the processes and experimental tools cited by the literature is its inability to readily measure the outcome of such biological events or processes in a near real time method, as all the above procedures are dependent on secondary and lengthy analyses employing techniques such as PCR or ELISA for assessing the outcome of the experiments, while the proposed method of isothermal gene editing employing the CRISPR/Cas9 methodology of cutting and splicing and replicating a DNA segment of interest and altering the DNA structure is supplemented by exploiting the properties of the conjugated gold nanoparticles (GNP) to the a specific locus of a gene of the DNA and marking such locus with a GNP acting as a mass enhancement. Such conjugation to the specific gene in question is immediately available to the detector with analysis by using the SH SAW biosensor. The detector and its reader measure the resultant conjugation of GNP and gRNA at the specific site. By use of this technique, the detector electronics records and displays such biochemical events. The system register then reports the resultant output of these biological events as a corresponding change in frequency, or phase-shift.

The step of conjugating a nanoparticle to the selected protein includes the step of conjugating a gold or iron nanoparticle to the selected protein.

The step of performing a CRISPR/Cas9 preparation of the DNA segments to cut and splice a selected protein into a selected target site on at least one of a plurality of the DNA segments includes the steps of: functionalizing the detection surface of the SAW with streptavidin; binding a first Cas9 protein with a first guide RNA to a first selected target site on at least one of the plurality of DNA segments, the first Cas9 protein and the first guide RNA comprising a first RNA/Cas9 pair; biotinylating the Cas9 protein; binding the biotinylated Cas9 protein to the streptavidin; and binding the selected protein as a second Cas9 protein with a second guide RNA to the second selected target site on at least one of the plurality of DNA segments, the second Cas9 protein and the second guide RNA comprising a second RNA/Cas9 pair.

The method further includes the step of disposing a semiconductive layer on the nanoparticle, wherein measuring the number of DNA segments with conjugated nanoparticles using a surface acoustic wave sensor (SAW) comprises utilizing an electromagnetic property of the semiconductive layer to measure the number of DNA segments with conjugated nanoparticles.

The step of disposing a semiconductive layer on the nanoparticle comprises disposing a selectively doped semiconductive layer on the nanoparticle so that an active or passive electrical device is formed in the semiconductive layer.

The step of disposing a selectively doped semiconductive layer on the nanoparticle so that an active or passive electrical device is formed in the semiconductive layer includes the step of forming an antenna, diode or transistor in the semiconductive layer.

The scope of the illustrated embodiments of the invention also include a delivery system for use in a surface acoustic wave sensor (SAW) for isothermal detection of DNA including a plurality of delivery vehicles, each including (i) one or more guide RNA (gRNA) and (ii) a nucleic acid editing system. One or more gRNA is provided in a first delivery vehicle and the nucleic acid editing system is provided in a second delivery vehicle including a conjugated gold or iron nanoparticle, so that a limit of detection of the surface acoustic wave sensor (SAW) is improved.

The delivery system further includes an active or passive selectively doped semiconductive layer disposed on the gold or iron nanoparticle; so that electromagnetic interaction with the semiconductive layer is utilized in the surface acoustic wave sensor (SAW) for detection.

The illustrated embodiments of the invention also include a method comprising the step of modifying a target nucleotide sequence in a DNA segment to enhance the mass of the DNA segment for detection by a surface acoustic wave sensor (SAW). The method includes the step of administering to the DNA segment a delivery system for isothermal detection of DNA including a plurality of delivery vehicles, each including (i) one or more guide RNA (gRNA) and (ii) a nucleic acid editing system, wherein the one or more gRNA is provided in a first delivery vehicle and the nucleic acid editing system is provided in a second delivery vehicle including a conjugated gold or iron nanoparticle, so that a limit of detection of the surface acoustic wave sensor (SAW) is improved.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a molecular diagram of a DNA segment which has been modified by using the CRISPR/Cas9 methodology to enhance the mass of the segment with a conjugated nanoparticle.

FIG. 2 is a molecular diagram similar to FIG. 1 wherein the number of CRISPR/Cas9 pairs has been multiplied to obtain an amplified mass enhancement.

FIGS. 3a and 3b are molecular diagrams where the CRISPR/Cas9 methodology is used to provide for control of a nonspecific signal. FIG. 3a shows the use of a second and third CRISPR/Cas9 pair on a DNA segment. FIG. 3b shows the cutting of the DNA segment by the second CRISPR/Cas9 pair.

FIG. 4 is simplified diagrammatic representation of a biosensor where a full-length antibody is captured by an organosilane linker on silica dioxide for immobilizing protein in solution.

FIG. 4a is a surface acoustic wave device with a single delay line, fabricated on a piezoelectric substrate. The output signal is compared to the signal from a reference lane and the phase, frequency or amplitude differences determined using a mixing cell.

FIG. 4b is a diagram of the sensing surface of a shear wave acoustic biosensor where a coating and guiding layer as used to form boundary conditions for the acousto-electric wave transmission induced in a sensing layer from an adjacent fluidic medium.

FIG. 5 is a molecular diagrammatic model of the functionalization of SAW with silicon layer (Si), Silicon Oxide layer (SiO₂), LiTaO₃ (piezo) layer, chemically functionalized layer including a linker molecule, spacer molecule, antibody fragments and an exemplary analyte protein.

FIG. 5a is a schematic representation of a SAW sensor with the sensing area functionalized as depicted in FIG. 5.

FIG. 5b is a schematic representation illustrating the combination of the SAW sensor with a phase shift circuit and amplifier.

FIG. 5c is a schematic representation of a dual SAW sensor providing a sensing lane and reference lane configured with a common RF source and a common processing unit.

FIG. 6 is a diagrammatic representation of the array of SAWs cell units each configured with a source follower amplifier.

FIG. 7 is a block diagram of a pathfinder/reader describing the analog front end coupled to the array of cells that are multiplexed and digitized by its microcontroller and peripherals.

FIG. 8 is a block diagram of the electronic circuit, for the detection, analysis and data processing of the SAW biosensor.

FIG. 9 is a flowchart detailing the auto gain selection software logic designed to select the proper post-amplifier gain based on the saturation detection circuit output to insure the impedance signal while using impedance converter line.

FIG. 10 is one example of a schematic representation of an analog front end (AFE) with selective connectivity to the analog processing platform.

FIG. 11 is a schematic depiction of a shear horizontal wave and a droplet of liquid loading the same.

FIGS. 11a and 11b are schematic diagrams of functionalized DNA segments illustrating the phase shift with mass loading in FIG. 11a and without mass loading in FIG. 11b.

FIG. 12 is a graphic representation of the integrated microfluidic chamber with enhanced convection flow to the sensor(s) array.

FIG. 12a is a schematic representation of the integrated microfluidic chamber flow diagram with its peristaltic pump.

FIG. 13 is an orthographic representation of the Euler angles, a means of representing the spatial orientation of the crystal LiTaO₃ with its Y cut and its wave-propagation reference frame (coordinate system or basis) as a composition of three elemental rotations starting from a known standard orientation of 36° relative to its x-axis frame.

FIG. 14 is a diagram of a SAW device having its input coupled to an RF source and its output to a signal processing unit.

FIG. 15 is a representation of a top-level architecture of the SAW system electronics.

FIG. 15a is a block diagram describing the phase measurement system using a digital I/Q demodulator.

FIG. 15b is a system diagram of the SAW analog front end with its IF electronic scheme.

FIG. 15c is an LTspice simulation with phase values before down-conversion at Ref/Sig @325 MHz, delta phase=90 deg, resulting in time domain of 0.77 ns.

FIG. 15d is an LTspice simulation with phase values after down-conversion at intermediate frequency with Ref/IF Sig @250 KHz, delta phase=90 deg, resulting in time domain of 1 μs.

FIG. 16 is a schematic representation of the digital I/Q demodulator circuit.

FIG. 17 is a schematic representation of current-voltage (I-V) and dI/dV vs Vsd curves for a Au—SiO₂ composite nanoparticle confined on a silicon substrate.

FIG. 17a is microphotograph of a single electron transistor (SET) employing a chemically modified GNP to form a Coulomb blockade in a tunnel junction.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

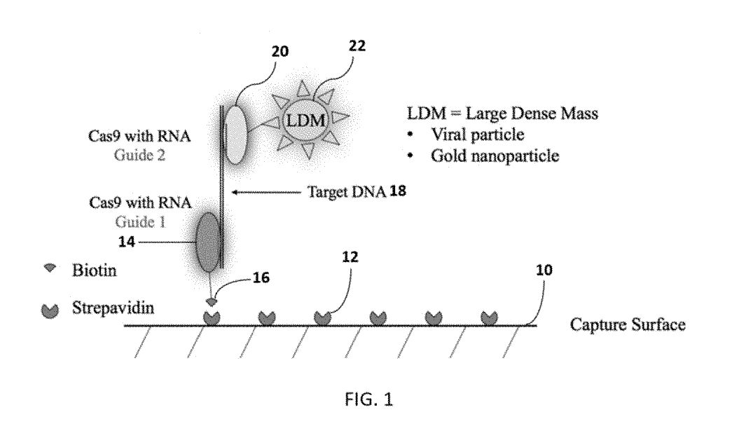

Using two Nickase dead mutant Cas9 proteins, each loaded with different guide RNA, a DNA capture and detection amplification system is developed for DNA detection in a SAW device. No temperature cycling is required in preparation of the DNA segments as is the case of polymerase chain reactions (PCR). A Cas9 protein with guide RNA 1, collectively denoted by reference numeral 14, is modified such that it can be captured on the surface of a SAW chip 10 as diagrammatically depicted in FIG. 1. For example, the detection surface of SAW chip 10 is coated with strepavidin 12 to bind biotinylated Cas9 14 using biotin 16. The guide 1 RNA 14 encodes for binding to a selected DNA sequence 1 and hence any DNA 18 containing this sequence will be captured on the chip surface 10 by the biotinylated Cas9 14. The strepavidin/biotin system 12, 16 is only one example of potential capture pair which could be utilized, and it has the advantage of binding to surface 10 such that DNA 18 tends to be vertically oriented or orthogonal to surface 10. This allows for higher density bindings on surface 10 as well as an optimized orientation for viral particle capture.

Reducing LOD by Incorporating GNP

A second CRISPR-Cas9 pair with guide 2 RNA, collectively denoted by reference numeral 20, provides signal amplification and orthogonal specificity. The guide 2 RNA 20 for the detection complex encodes for a second selected independent DNA site (DNA sequence 2) near the first site (DNA sequence 1). The distance between the two binding sites is anticipated to be less than 100 nucleotides, as determined empirically. The second Cas9 20 is modified so that it is tethered to a large, dense mass (LDM) 22. In the illustrated embodiments the mass 22 comprises a “nanoparticle”, but it is within the scope of the embodiments of the invention that it could include a bacteriophage virus such as PhiX174 or be a (gold) nanoparticle. The composition of the nanoparticle mass 22 is determined empirically in SAW experiments. The enhanced mass 22 allows the limit of detection of the SAW to be substantially increased. For example, with a low cost electronic implementation of the SAW, the limit of detection can be reliably reduced to an order to 10-100 DNA strands.

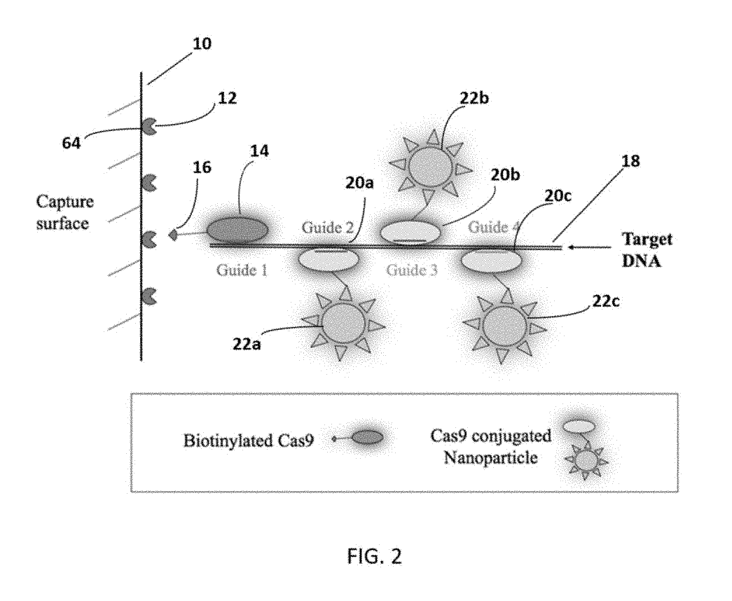

Variations on this basic two component system includes additional detection CRISPR-Cas9 pairs to increase signal amplitude as well as specificity by using the modifications as diagrammatically illustrated in FIG. 2. A first CRISPR-Cas9 pair 14 and second CRISPR-Cas9 pair 20a with LDM 22a are employed and bound to DNA 18 in the same manner as described above about FIG. 1. Incorporation of a plurality of additional CRISPR-Cas9 pairs 20b and 20c with LDMs 22b and 22c respectively, for example, provide amplified mass enhancement further boosting the limit of detection of the SAW device.

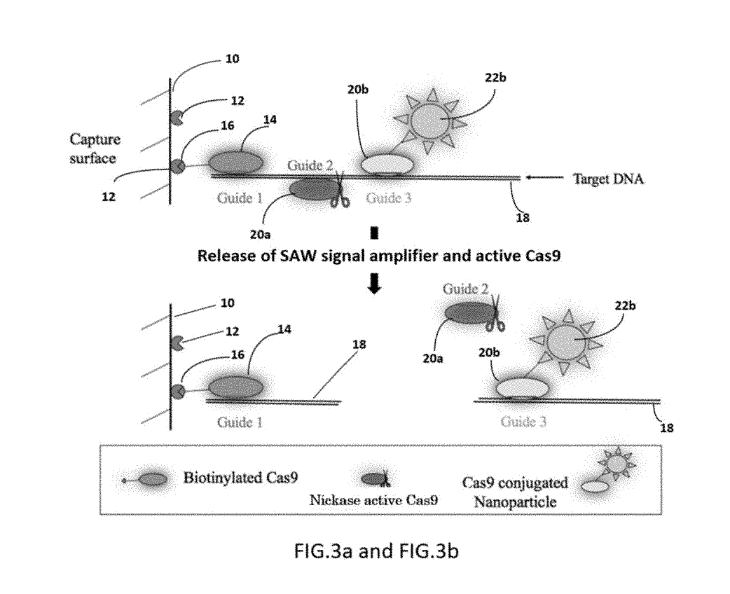

Further, incorporation of a third CRISPR-Cas9 pair 20b that is DNA cleavage competent allows release of the third detection pair after a measurement. This would control for a nonspecific or negative signal as the removal of the Cas9 with guide 2 RNA 20a, indicating that the specific gene modified site, is no longer present at the sensing lane substrate 10, therefore the measuring surface (sensing area 10) will contain only the remaining Cas9 with guide1 RNA 14 attached to linker molecule (biotin) 16 which is conjugated to the surface chemistry modifier (streptavidin) 12 as diagrammatically depicted in FIGS. 3a and 3b. FIG. 3a diagrammatically illustrates a second and third CRISPR-Cas9 pair 20a and 20b respectively. CRISPR-Cas9 pair 20b includes a conjugated nanoparticle 22b. CRISPR-Cas9 pair 20a is cleavage competent so that after a measurement of DNA 18 with both second and third CRISPR-Cas9 pairs 20a and 20b attached to DNA 18, DNA 18 can be selectively cleaved by second CRISPR-Cas9 pair 20a, thereby releasing CRISPR-Cas9 pair 20b and its conjugated nanoparticle 22b, which allows for the specific detection of the indicated target of CRISPR-Cas9 pair 20a.

Another variation allows for the detection of a single nucleotide polymorphism by using a fine-tuned guide RNA sequence specifically targeted for the nucleotide polymorphism of interest.

While the earlier discovered CRISPR/Cas9 system was used as an example, subsequently uncovered systems may prove more advantageous due to more desirable biochemical traits.

Overall, this is the first system to detect sequence specific DNA at or below a hundred-molecule level, at a constant room temperature with no thermocycling (PCR), in a few minutes.

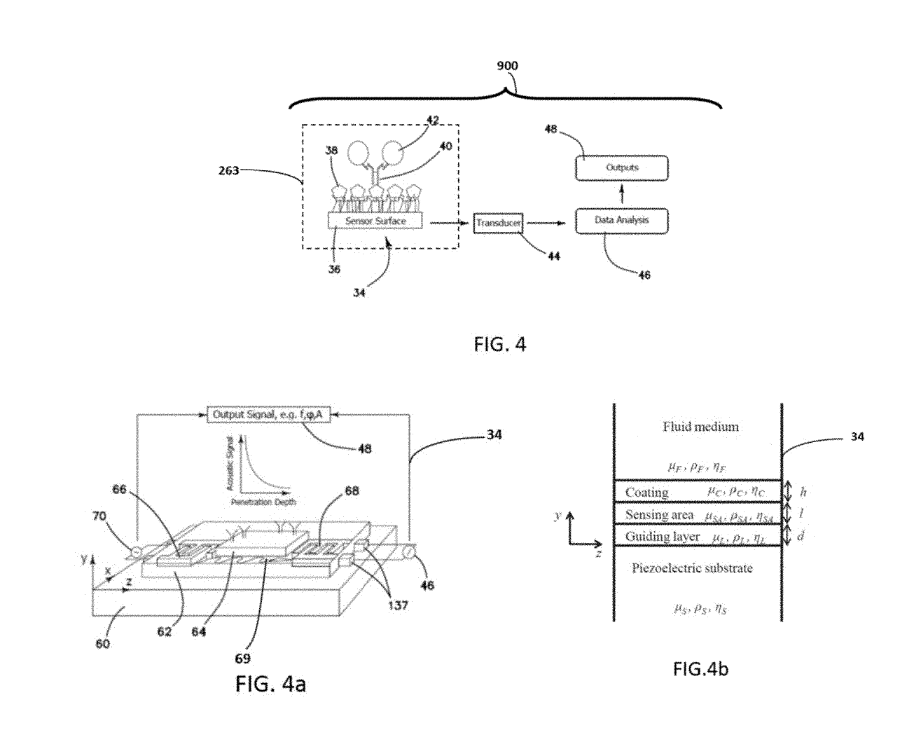

The operation of the SAW device of FIG. 4 is based on acoustic wave propagation near the surface of piezoelectric solids diagrammatically depicted in FIG. 4A. A piezoelectric substrate 60 has a guiding layer 62 formed thereon in which there is an input interdigitated transducer (IDT) 66 and output interdigitated transducer (IDT) 68 in FIG. 4a, the latter comprising transducer 44 of FIG. 4. Between IDTs 66 and 68 is a chemically sensitive layer 64, which may be a functionalized gold layer. An RF driver 97 creates the SAW waves in the guiding layer 62 by means of IDT 66, which are then transmitted or launched into sensitive layer 64, which will be loaded by the detected analyte (Shown in FIG. 1 as streptavidin 12, attached to linker biotin 16 and target DNA 18 with its Cas-guide 1 RNA 14 supplemented by the large dense mass 22 (GNP)). The output IDT 68 is excited by the modified SAW wave, and transduces it into an electrical signal, which signal is detected by data analysis circuit 46 of FIG. 4 and then communicated as a collective output signal 48 as the frequency, phase and/or amplitude of the modified SAW wave. The RF signal from RF source 97 may also be utilized as part of the collective output signal 48. The wave can be trapped or otherwise modified while propagating in a traverse mode in layer 64. The displacements decay exponentially away from the surface, so that most of the wave energy is confined within a depth equal to one wavelength.

The interdigital transducers 66, 68 are comprised of a series of interleaved electrodes made of a metal film deposited on a piezoelectric substrate. The width of the electrodes usually equals the width of the inter-electrode gaps (typically ˜0.3 μm) giving the maximal conversion of electrical to mechanical signal, and vice versa.

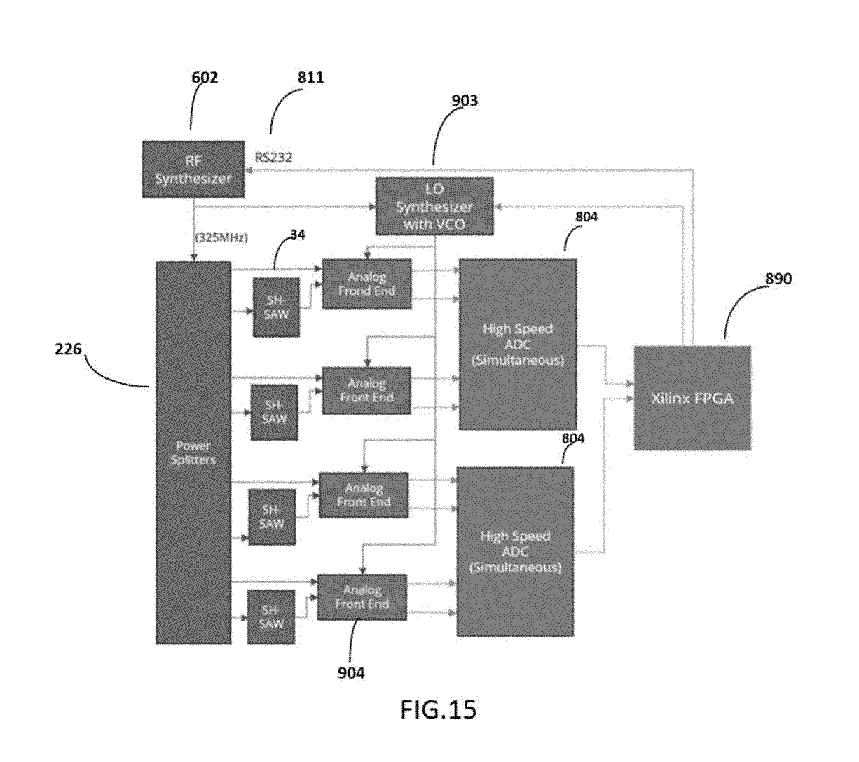

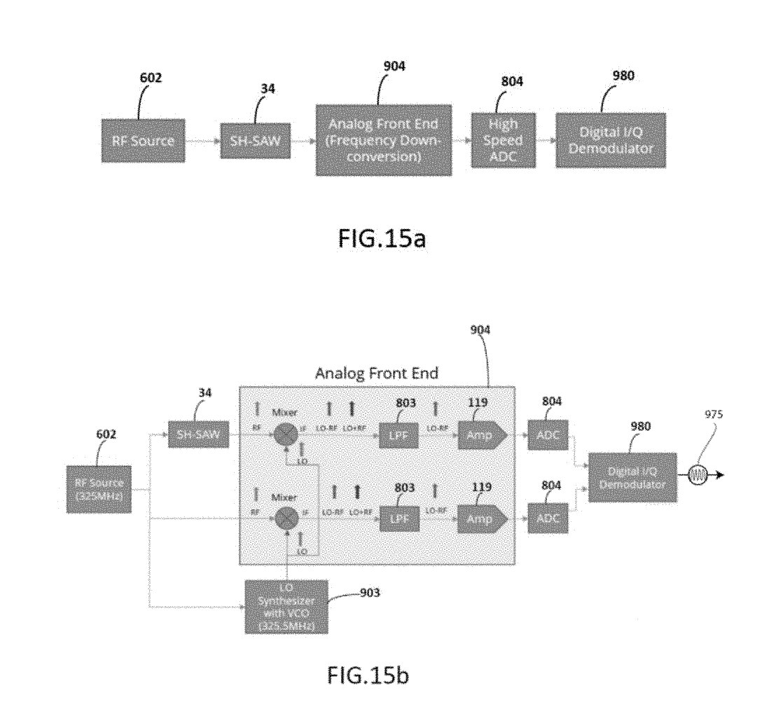

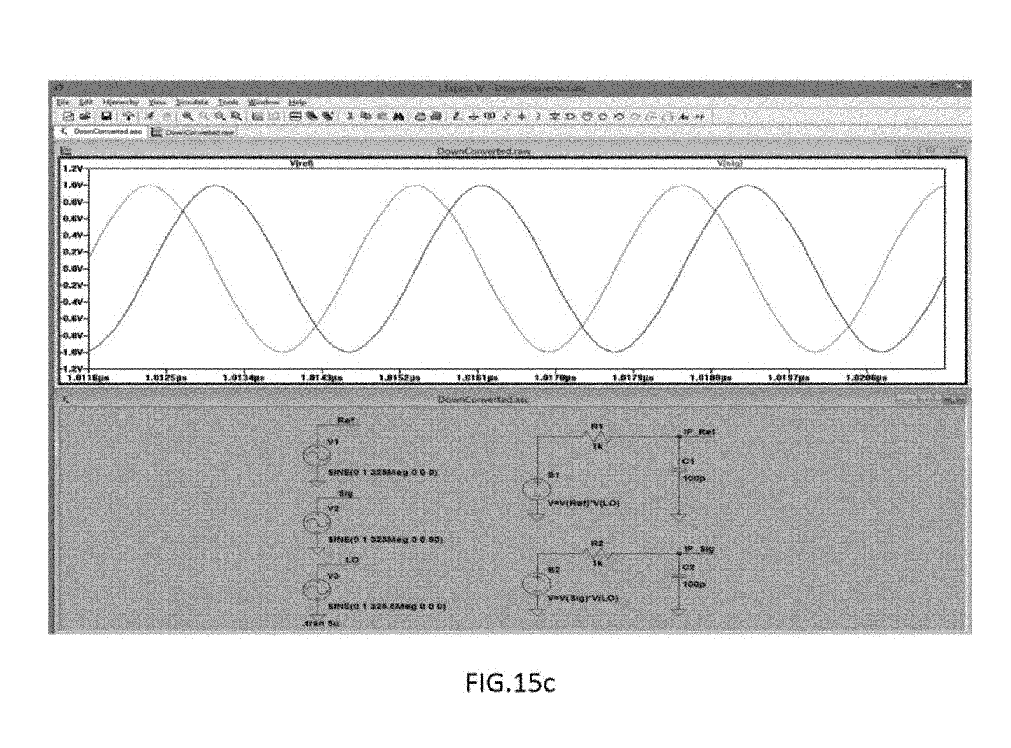

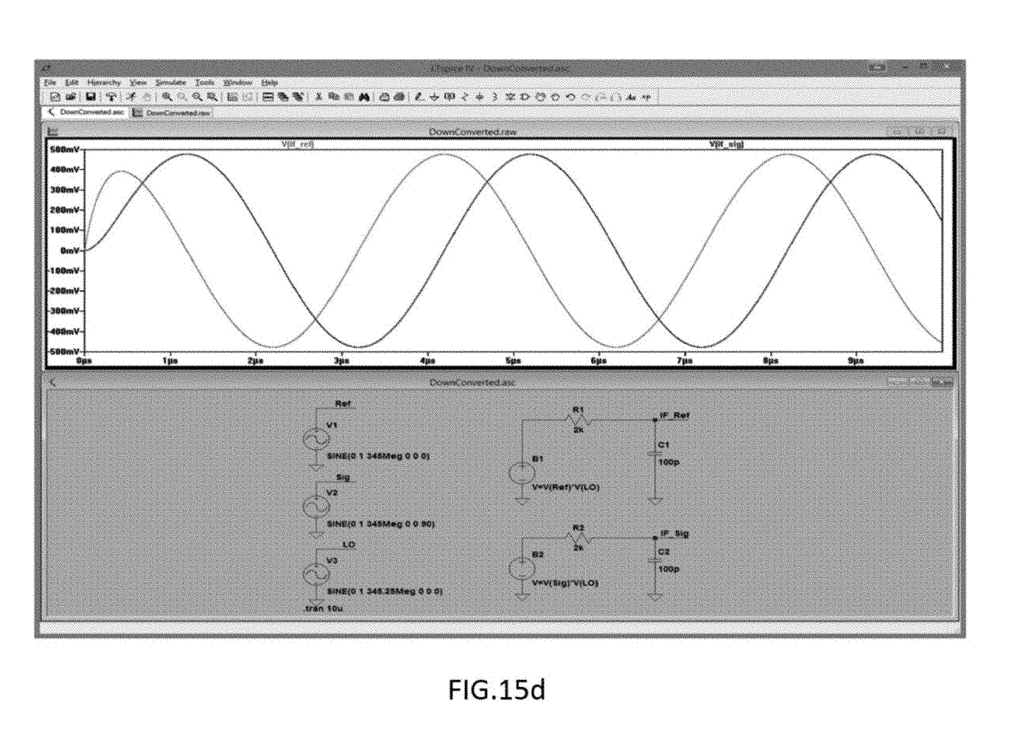

Controlling the covalent bonding of antibodies 12 and 16 onto functionalized substrate 64 using a SH SAW platform 500 is a key step in the design and preparation of label free-based transducer for targeting cells, biomarkers and synthetic oligonucleic acid or peptide or DNA 18. The chemical biosensors forming the sensing substrate 64, their chemical probes and the sensing/reference lane's architecture, undergo conformational electrical impedance (phase shift) changes due to hybridization of bioagents. The changes are realized through the respective mass-loading (in the time domain) with kinetic detection by electronics as described in FIGS. 14, and 15a-15c.

Two devices of the type illustrated in FIG. 4a are provided giving a total of two pairs of IDTs, one pair employed as a sensing lane and the other as a reference lane. The SH SAW transducer 44 functioning as a sensing lane is controlled by the mass-loading of the analyte 42 and its targeted probe 40 (namely an antibody, an antigen, a protein, a receptor, an aptamer, a peptide, a DNA strand, or an enzyme), or by hybridization of the analyte to its specific antibody, thereby changing the mass on the sensing lane 64. The device functioning as a reference lane generates a relatively stable and unchanging signal due to the use of nonspecific antibody such as IgA, IgD, IgE, or IgG. The illustrated embodiments center on the ability of the transducer's design to control the boundary conditions parameters, thereby leading to improved parameters of the SAW biosensor system. The contributing effects that improve the signal-to-noise ratio (SNR) of the resultant measured output is manifested in the phase shift in the frequency domain and its time constant. The optimization of such an apparatus is determined by the variability and characteristics relating to the crystal type e.g. LTaO₃ with a cut defined as 36° Y cut and X-axis wave propagation, waveguide geometry and metrics, waveguide delay layer e.g. the use of SiO₂ dielectric, microfluidic acoustic properties and its hydrostatic flow rate, analog front-end circuitry, interdigitated electrode and impedance matching value, and frequency domain value, e.g. 375 MHz.

Turn now and consider chemical functionalization of the biological probe. See the example shown in FIG. 1 as streptavidin 12, attached to linker biotin 16 and target DNA 18 with its guide 1 Cas-RNA 14 supplemented by the large dense mass 22 (GNP). All the above is an illustration of the steps taken by this application for reducing or minimalizing the LOD, thereby increasing sensitivity of the apparatus. This task of minimizing error associated with the design and fabrication of the SAW platform illustrated in FIG. 4a is further complicated by the fact that a label free, near real time response of such a device, for the detection of an antibody, an antigen, a protein, a receptor, an aptamer, a peptide, a DNA strand, or an enzyme requires a careful analysis of the contributing factors, which impact the resultant phase shift because of the mass loading on the sensing lane must account for the covalent binding of the analyte to its antibody without the secondary effects of ionic contamination, SNR and data acquisition. These issues render the prior art solution to the problem of fabricating a SAW device with resolution and accuracy on the par with PCR and ELISA unachievable. The illustrated embodiments solve some of the limitations of the prior art by sorting the primary contributing factors which enables us to detect and reliably measure the degree and time-sequencing of a plurality of biomarkers in a hand-held device integrated with a microfluidic chamber and its associated electronic reader.

One of the preferred embodiments of this application is illustrated by the ability of the system shown in FIGS. 4 and 4a, and the use of an isothermal DNA editing (shown in FIGS. 1, 2, 3a and 3b to enable a detection methodology of full-length antibodies captured by an organosilane linker on silica dioxide for immobilizing protein in solution, where a monolithic SAW biosensor system is fabricated on a lithium tantalite substrate (LiTaO₃) that utilizes the strategies of mass enhancement where a GNP is added to the specific gene of a DNA and where such a mass provides the minimum threshold necessary for the SAW to detect the analyte in question. The process outlined in FIG. 1 for conducting near real-time detection of pathogen or disease biomarkers is coupled with an electrochemical system for rapid nucleic acid analysis process as described in the figures.

Gene Editing Detection Employing SAW

In one of the preferred embodiments we describe an isothermal DNA detection methodology employing a surface acoustic wave, where a gRNA coupled with a GNP form a conjugate with specificity and minimal mass threshold to meet the objective of minimizing the LOD. It should be noted that the use of mutated “nickase” version of the Cas9 enzyme generates a single-strand DNA break (Nick) at a specific location based on a co-expressed gRNA-defined target sequence, thereby reducing potential error of non-specificity. Such homology improves the odds of the detector (the SAW) to generate a statistically high reliability signal response with minimal LOD, since the minimal thresholds of the system sensitivity rely on the GNP payload and its added mass.

In one of the preferred embodiments the SAW biosensors are specifically targeted towards the detection of DNA of whole microbial pathogens, DNA of protein biomarkers and nucleic acid in a biological matrix. The illustrated SAW biosensors provide relevant information for patients who are likely to respond to a given therapy, as well as biomarkers that can measure a patient's response to therapy. In one of the embodiments of this application we teach how these two measures are necessary for personalizing the drug treatment for each patient. The SAW devices will provide information with respect to rapid, point-of-care detection of biological contaminations or infection.

In one example, we teach the use of the method in the field of food safety, namely monitoring food quality before food items are consumed.

The use of interdigitated electrodes with acoustic wave biosensors on a lithium tantalite piezoelectric-substrate presents a platform which is ideally suited as a label-free biosensor for aqueous-based samples. This general technology of SAW has been used extensively as a portable, rapid and sensitive detection system for decades, primarily in the telecommunications industry. Recent advancements in the technology, has allowed for the creation of guided shear surface acoustic wave (SAW) devices that can operate in liquid environments. These devices can be functionalized through immobilization of antibodies or antibody fragments to target biomarkers and enhanced using the isothermal DNA detection while adding a GNP as a mass enhancement to improve the use of this system over previous generation of SAW devices. This involves enhanced surface chemistry techniques along with methods of attaching antibody fragments that bind specifically to biomarkers for various diseases, a novel method described in FIGS. 1, 2, 3a and 3b.

When performing SAW biosensing in liquid environments for the detection of microbes, eukaryotic cells, protein biomarkers or nucleic acid sequences, there is a strong loss of longitudinal bulk modes such as Raleigh surface waves and most Lamb-wave modes. Surface waves with displacements normal to the surface generate compressional waves, which dissipate wave energy in the liquid. For this reason, acoustic waves that have the particle displacement parallel to the device surface and normal to the wave propagation direction are essential. These waves, which are referred to as shear-horizontal (SH) waves, propagate without coupling acoustic energy into the liquid. Love-waves are SH waves that propagate in a thin guiding layer on SH-SAW devices. If a no-slip boundary condition is assumed at the sensing surface, a thin layer of liquid becomes entrained with a shear movement at the surface for viscous liquids. This viscous loading affects the Love-wave in two ways. First, the entrainment results in mass loading of the wave-guiding layer, resulting in changes to the wave number. Second, the wave becomes damped due to viscous losses in the liquid. To reduce aqueous effects, the guiding layer can be shielded with suitable dialectic material e.g. SiO₂, to prevent electrical loading on the IDTs. Love-waves that are SH are confined to the thin layer between the wave-guides with the requirement that the shear velocity in this guiding layer (the velocity in the material forming the wave-guides) is less than the shear velocity in the piezoelectric substrate. For this reason, the waveguide is the most significant structure for proper Love mode operation as a mass-sensitive biosensor.

SH SAW devices are well known to offer high surface-mass detection sensitivity for biochemical sensing. It is possible to measure mass sensitivities from surface loading in the 1-100 ng/cm² range. The traditional configuration of SAW devices involves a chemically functionalized area that immobilizes a targeted species with a selective surface coating. The novel use of attaching the targeted species via the gRNA+GNP perturbs the mass of the sensing lane by providing a beacon with added mass at site specific area defined by the attached gRNA. A propagating surface acoustic wave is generated by the interdigitated (IDT) gold or aluminum electrodes, and such a wave responds to the added mass with a proportional electrical phase shift output. The system uses a reference lane, which employs an antibody that is nonspecific to the target. This reference lane (with similar dimensional footprints and matching impedance) is used to enable a differential mode for measuring the binding between the specific added mass and the reference, thereby eliminating the needs for absolute or calibrated standard.

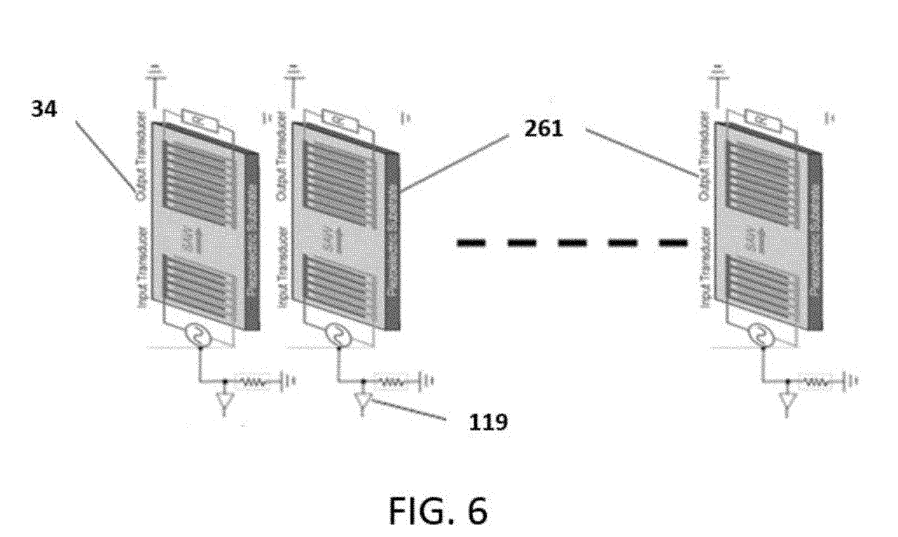

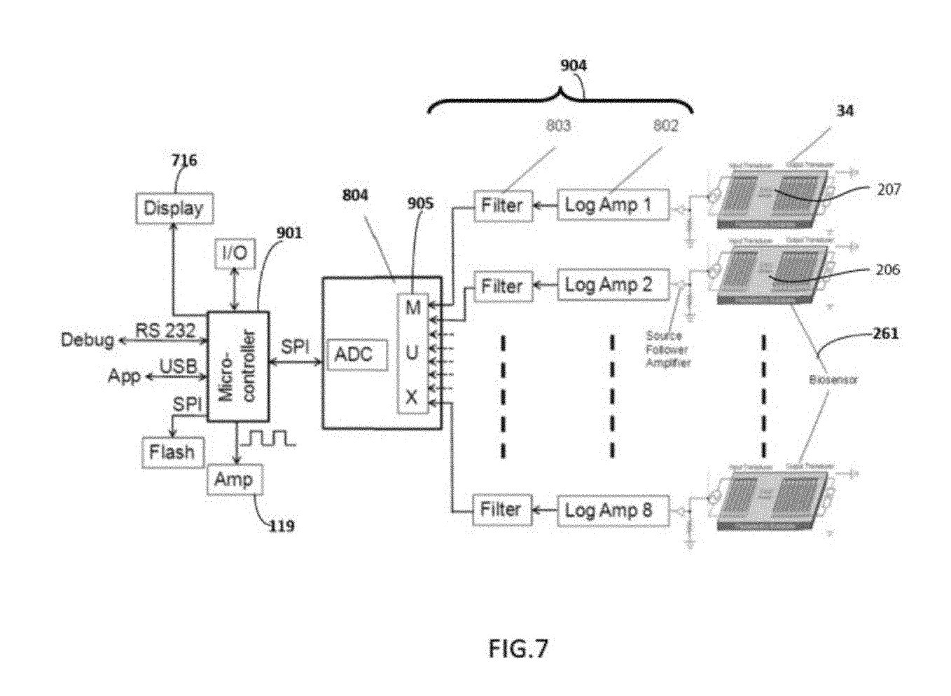

The acoustic wave is detected by a second set of IDTs 68 located across from the first set of IDT 66. If the targeted species is present, then the propagating wave will be perturbed in such a way to cause a shift in the phase, frequency or amplitude, relative to wave that propagated across the reference electrode. This configuration is illustrated in FIG. 4a. These miniature biosensors are often used in an array format (shown in FIGS. 6 and 7), where multiple delay lines can be scanned for a single target in parallel, but the device could also be operated in series to scan for multiple biomarkers for different diseases. Operating a SAW array allows rapid, point-of-care diagnostic using small portable devices. The use of miniature monolithic SAW sensor arrays allows on-chip signal processing, and allows the chips to be fully integrated into a larger system and can be easily packaged.

Temperature compensation is inherent to this system since the SAWs reference and the sensing lanes are on the same substrate and therefore experience the same temperature fluctuations. Therefore, any adjustment to the phase due to temperature fluctuation is automatically adjusted and compensated. The determination of the phase shift is defined by using homodyne mixing using a Gilbert cell mixer, (illustrated in FIG. 15b where that the two-differential amplifier are formed by emitter-coupled transistor pairs whose outputs are connected (currents summed) with opposite phases). The mixer extracts the difference in phase between the resonance frequency from the reference and the resonance frequency of the delay line being probed. The resulting phase shift is then calibrated for changes in the mass loading of the surface. This process cancels any temperature dependence. Measurements in all lanes are differential measurements relative to the reference lane. Both the reference and the delay lanes experience the same changes since the reference lanes are essentially a built-in control.

In one embodiment, the SAW device with a single delay lane, fabricated on a piezoelectric substrate such as LTaO₃, obtains a differential output signal between the sensing and reference lanes, where phase, frequency or amplitude differences is determined using a mixing cell. With the electronic scheme described in FIGS. 15c and 15d a marked improvement of the resulting signal is realized. An LTspice simulation of the system's phase-value is graphically illustrated before down-conversion at Ref/Sig at 325 MHz with delta phase=900, equal to 0.77 ns time scale, and results of the signal is compared with the output signal illustrated by FIG. 15d. An LTspice simulation of the improved time delay is shown, where the phase-value is generated after down-conversion is performed with an intermediate frequency (IF) where the Ref/IF Sig at 250 KHz is displayed with delta phase=90°, resulting in 1 μs time scale where the frequency and phase are preserved, but the time scale is set to an order of magnitude lower. The technique of time delay conversion is essential to the ability of the sensor electronics to perform the analog to digital conversion, and enable capture as well as analysis of the data generated without any loss of magnitude or phase.

In one embodiment of this application we teach the value of employing the improved technique of gRNA conjugation of GNP to the specific gene, where we employ an antibody-based coated SAW biosensor which permits the rapid and sensitive analysis of a range of pathogens and their associated toxins. The presence of bacterial pathogens, fungus and viral particles are ubiquitous in our environment and can pose considerable risk to persons, who are exposed to these pathogens. Therefore, monitoring for the presence of microorganisms will be critical for maintaining proper health. This is especially true for short shelf-life foods.

Mass-based piezoelectric biosensors operate on the principle that a change in the mass, resulting from the molecular interactions between a targeting molecule and the target analyte, can be determined. For example, mass changes result in alterations in the resonance frequency of a lithium tantalite crystal. These piezoelectric sensors are affordable and disposable options for pathogen and biomarker detection.

In one embodiment, we employ the improved technique of DNA gene's editing for detecting the outbreaks of food-borne and water-borne pathogens, which remain a major cause of disease and mortality throughout the world. The rapid detection of these pathogenic microorganisms is critical for the prevention of public health epidemics. The quantitative identification of microorganisms has become one of the key points in areas of biodefense and food safety. To date, the detection and identification of pathogens rely primarily on classic microbiology methods of culturing. In such cases, the technician is required to go through a series of handling steps. There are several rapid methods that are now also used in microbiology that utilize enzyme-linked immunosorbent assays (ELISA) or polymerase chain reaction (PCR) assays. These methods are laborious and time consuming. These methods are also not able to deliver real-time analysis or point-of-care or detection's analysis. The use of portable biosensors to rapidly identify pathogens in food and water offers several advantages over the other rapid methods. The use of the isothermal DNA employing crisper Cas9 methodology as is described by the application, facilitate such findings and enable a near-real time analysis.

Experimental Results of SAW Performance

In one embodiment of this application we employ the use of the SAW sensor disclosed in U.S. Pat. No. 8,436,509 Branch et al, incorporated herein by reference in its entirety, where the author describes a study of low-level detection using a SH SAW biosensor to identify a pathogen such as Bacillus Anthracis Simulant. The study with its Love-Wave biosensors, employed a 36° YX LiTaO₃ piezoelectric substrate, with physical and electrical characteristics like the one employed by this application. The study demonstrated results in which the device detected an endospore from the gram-negative bacteria specie (Bacillus thuringiensis B8). Bacillus anthracis is the causative agent for anthrax, a potentially fatal bacterial infection that has being used as a bioterrorist agent. The results indicate that the level of detection is equal to the control group tested by polymerase chain reaction (PCR method).

In other studies, such as reported in Branch et al, “Low-level detection of a Bacillus anthracis simulant using Love-wave biosensors on 36°YX LiTaO₃”, the sensor (SH-SAW biosensor), was used to demonstrate a detection level of 1 ng/cm² when using a polyimide guided layer and BSA as the blocking agent. The detection of such low levels of anthrax simulants revealed the ability of the sensor platform to detect clinically relevant doses of anthrax. The authors employed a similar device to detect both HIV1/HIV2 and Ebola viruses at clinically relevant doses. This application employs a similar configuration of the sensor architecture to obtain improved results by the incorporation of DNA editing technique noted and illustrated by FIGS. 1, 2, 3a and 3b.

In preliminary experiments noted by the of U.S. Pat. No. 8,436,509, the SAW biosensor proved capable of detecting concentrations spanning three orders of magnitude, with an estimated limit of detection (LOD) of 74 cells were used. While the cell mass is not a true concentration, the use of an accurate standard curve will allow the rapid correlation to a potential start concentration. In these studies, the SAW biosensors, have been used to detect Ebola virus as a potential point-of care diagnostic tool. The area of improvement in such studies were limited by the inability of the prior art to reduce the LOD to a minimal threshold-relative to the SNR base, (Sensitivity vs. LOD), value of the system.

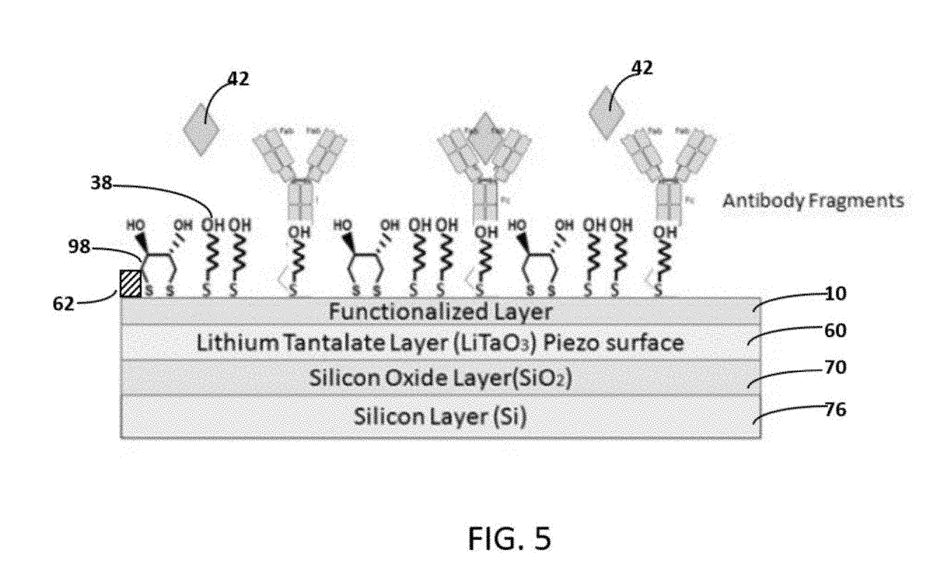

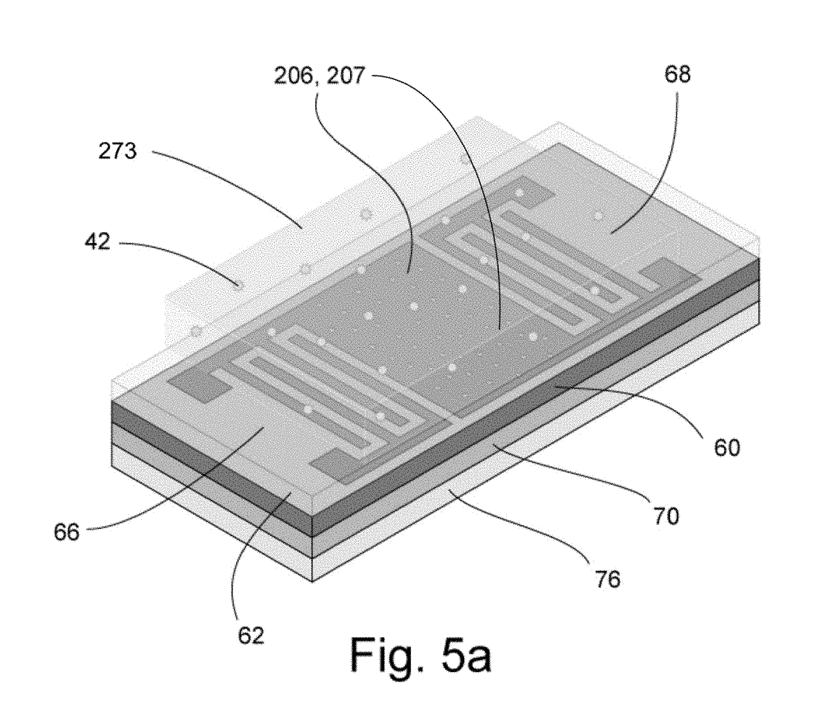

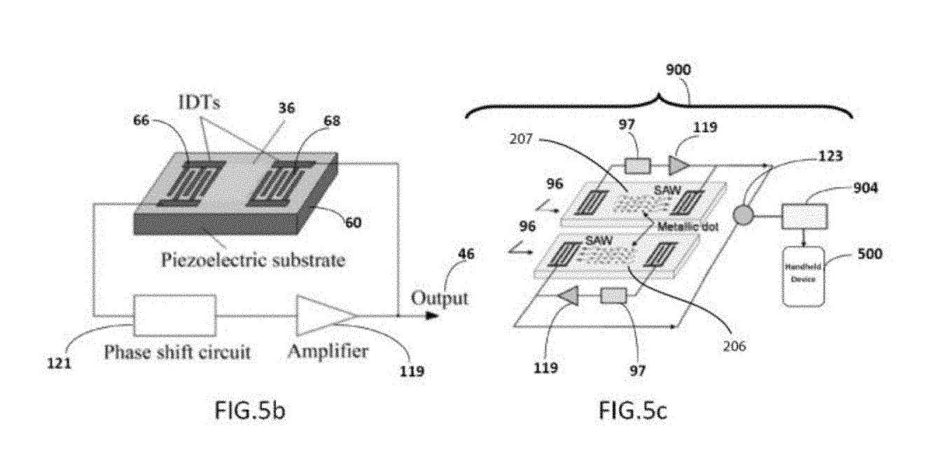

To achieve high sensitivity in SAW sensors thereby reducing the LOD to its minimal base line above SNR floor, and relative to the sensor frequency domain, it is essential to confine a maximum amount of acoustic energy near the surface of the substrate and minimize wave scattering into the bulk of the substrate. To achieve this in Love-mode SAW devices, a waveguide layer shown in FIG. 5A is employed. The application follows the finding and experimental observations noted by Falk Herrmann et al, IEEE Transactions on Ultrasonics, Ferroelectrics, and Frequency Control, vol. 48, No. 1, January 2001, pp. 268-273. Where the waveguide is made of SiO₂ with thickness of up to 2.0 μm were deposited onto the entire wafer using plasma-enhanced chemical vapor deposition (PCVD). Dielectric value material is used to confine acoustic energy close to the surface of the devices. For high waveguide efficiency, the coated material should have a wave velocity less than that of the base piezoelectric substrate material. Dielectric materials such as silicon dioxide, parylene, polymethylmethacrylate are optional waveguide materials.

Optimization of SAW while Reducing LOD

One of the elements forming the boundary conditions of the sensor performance is the concentration of the analyte. At low-level detection, and where the limit of detection (LOD) of bioagents (in an aqueous environment), is limited, it is the threshold and mass-sensitivity which limits the ability of the existing art to meet clinical and relevant information while employing the SAW biosensor.

The optimization of the Love-Wave acoustic sensor, is enhanced by novel method of isothermal DNA editing using the Crisper Cas9 process, which provides a SAW system for reduced analyte concentration by performing the procedure noted in FIGS. 1, 2, 3a and 3b, whereby editing the DNA and supplanting the site specific with added mass (say GNP with 40 nanometer diameter, turns the single DNA or segment of such genetic material into a measurable electrical event, counted as a phase shift by the apparatus' reader.

The invention provides a method and apparatus for sensing the presence and/or the behavior of individual target DNA or molecules dissolved in a sample, wherein single nanoparticles is used in detecting individual target in DNA or molecule, with high temporal resolution. This is achieved through the detection of a slight shift in the SAW sensor and where the frequency attenuation, in an individual target DNA or molecule binds or detaches from the nanoparticle. Such individual binding event can be observed with the apparatus of the invention with enhanced sensitivity. Details of the electronic data capture and the problems of temporal limitation of the existing art are noted and proposed solution to such limitations as it affects the LOD are described in relation to FIGS. 14-16 below.

There are multiple parameters which affect the SAW biosensor LOD, evaluation of the SAW sensitivity Sₘ^{v}Sₘ^{v} measure: defined by the smallest concentration of a measurand that can be reliably measured by an analytical procedure of the SAW, is used as the metric when comparing the results of the method provided by this application.

In one embodiment, we employed the intrinsic parameter of “Sensitivity vs. LOD” metric to optimize the sensor ability to reduce the LOD to a minimum of surface mass density (σ) relative to 3 times the SNR value. The sensitivity parameter measures the effective performance of the sensor output, where wave velocity (v) attenuation to the accumulated mass (m) is assessed. This measure is the metric scale which this application utilizes to verify the improvements of the sensor performance relative to the minimum LOD of the system's performance.

Sensitivity vs LOD Measure

The sensitivity Sₘ^{v} measure is further represented by the derivative of the response (R) with respect to the physical quantity (analyte mass loading) to be measured as a phase shift (M):

$$s = {lim}_{Δ M \to 0} \frac{Δ R}{Δ M} = \frac{dR}{dM}$$

(It is possible to have different units of sensitivity depending on the used sensor response. e.g. for frequency output sensors, frequency (R=f), relative frequency (R=f/f₀), frequency shift (R=f˜f₀) and relative frequency shift (R=(f−f₀)/f₀) can be found by the reader, where f₀ is the non-perturbed starting frequency).

The sensitivity of the proposed SAW sensors gives the correlation between measured electric signals delivered by the sensor and a perturbing event which takes place on the sensing area of the sensor, where a high sensitivity measure (M) relates as a strong signal variation with a small perturbation change. Depending on the electronic configuration used (shown in FIG. 14) the electrical signal measured by the SAW device can be: operation frequency, amplitude (or Insertion Loss), and phase shift. From this signal and using the theoretical models noted above, the phase velocity and group velocity can be obtained.

When the sensor response R (ref. designator 46 in FIG. 5b) is the phase velocity, and the perturbing event, comprising of hybridization of the guide RNA 20 with its conjugated GNP 22, (shown in FIG. 1) on the sensing area 10, is the added mass (a variation of the surface mass density of the sensing area 64), and where the loading mass σ (where σ is a constant associated with the coating thickness h, and where ρ_{c} is the coating density addressed as the resultant K²), is the velocity change (due mass added) and the sensitivity S^{v}_{σ} measure of the SAW device at a constant frequency is given by:

$${s}_{σ}^{v} = \frac{1}{{v}_{ϕ 0}} \frac{\partial {v}_{ϕ}}{\partial σ} {\vert}_{f}$$

Where v_{φ} is the unperturbed phase velocity and v_{φσ} is the phase velocity after a surface mass change. In this sensor applications, the phase velocity shift must be obtained from the experimental values of phase or frequency shifts relative to the initial conditions of the sensor parameter K². For the closed loop configuration, where the experimentally measured quantity is the frequency, the frequency mass sensitivity S^{f}_{σ}, is defined as:

$${S}_{σ}^{f} = \frac{1}{{f}_{0}} \frac{df}{d σ}$$

For the open loop configuration, where the experimentally measured quantity is the phase, the phase sensitivity (also called gravimetric sensitivity) is noted by the measure S^{φ}_{σ}. such that in absence of interference is defined as:

$${S}_{σ}^{ϕ} = \frac{1}{{k}_{Lg} D} \frac{d ϕ}{d σ} = {S}_{σ}^{v}$$

Where D is the distance between input and output IDT and k_{Lz} is the wave-number of the Love mode, therefore k_{Lz}D is the unperturbed phase equal to φ₀, when initial calibration of the system is performed.

The relationship between the elements forming the sensor shown schematically in FIG. 5, where the sensor element is formed out of silicon layer 76, silicon oxide 70, crystal-piezo surface 60, functional layer (sensing area 10, guiding layer 62, are variables which must meet a strict metric value(s) to enable the IDT input 66, IDT output 68 to achieve a wave characteristic with low insertion lose and maximum energy transfer across the guiding layer 62, further resulting with an output (phase shift) which mimics the added mass over the sensing lanes with minimal LOD. To achieve such outcome this application, incorporate an example of the use of method for isothermal DNA detection with added GNP to reduce the limited of detection which enable the identification of biological species without the inherent limitation of minimum mass density threshold relative to the highest frequency in which the SAW is operating.

SAW Modeling as an Oscillator with Delay Line

FIGS. 5a, 5b and 5c describe the SAW in term of an oscillator where IDT input 66, IDT output 68, formed over crystal LiTaO₃ 60, with RF driver source 97, phase shift circuit 121, amplifier 119, mixer 123, and hand-held device 500, and the mass loading area of conjugated mass (gRNA+GNP) on the sensing lane 36 act as a delay line of the oscillator forming the system 900 in FIG. 5c. The operation of system 900, is simply as a delay line, or as frequency-determining element of an oscillator circuit in a closed loop configuration. Effectively, in an oscillator circuit the SAW device is placed as a delay line in the feedback loop of the RF amplifier. The task of the RF amplifier is to compensate the insertion loss (IL) generated by the delay line. The complete circuit oscillates at a frequency such that the total phase-shift in the entire loop is a multiple of 2π radians.

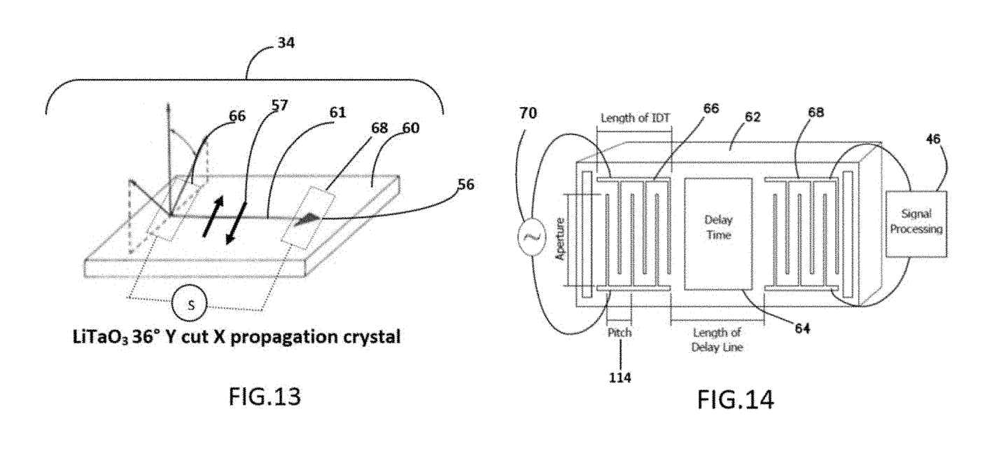

FIG. 13 is a graphic representation of the SH SAW waveguide sensor that operates with shear horizontal surface acoustic waves (SH-SAWs) are used for the characterization of liquids, including biological fluids. SH-SAW does not involve a normal component of mechanical displacement and exhibits weak damping when the wave-bearing surface contacts a viscous liquid medium, which makes it possible to use these waves in sensors to characterize liquids. In the case of SH SAW sensors, differences between the response signals to various analytes are achieved by using different thin film coatings in the SAW delay lines or using SAW delay lines at several different frequencies. SAW delay lines, which form the waveguide, impact the shear-horizontal waves propagating on the top layer of a coated layer, directly and reduce the acoustic noise. Attention to this element within the boundary conditions of the device must be focused on the selection of a material, which would effectively guide the Love wave. Silica and polymethylmethacrylate were used as guiding layers and the mass sensitivity of the corresponding sensors was tested in air.

The components described by FIGS. 13 and 14 indicated by the SAW with its wave propagation using the LiTaO₃ 60 with a crystal properties of 36° Y-cut and propagation along the X-axis direction 61. The delay line 64 produces a delay, T, which corresponds to the distance between the IDTs centers 66, 68.

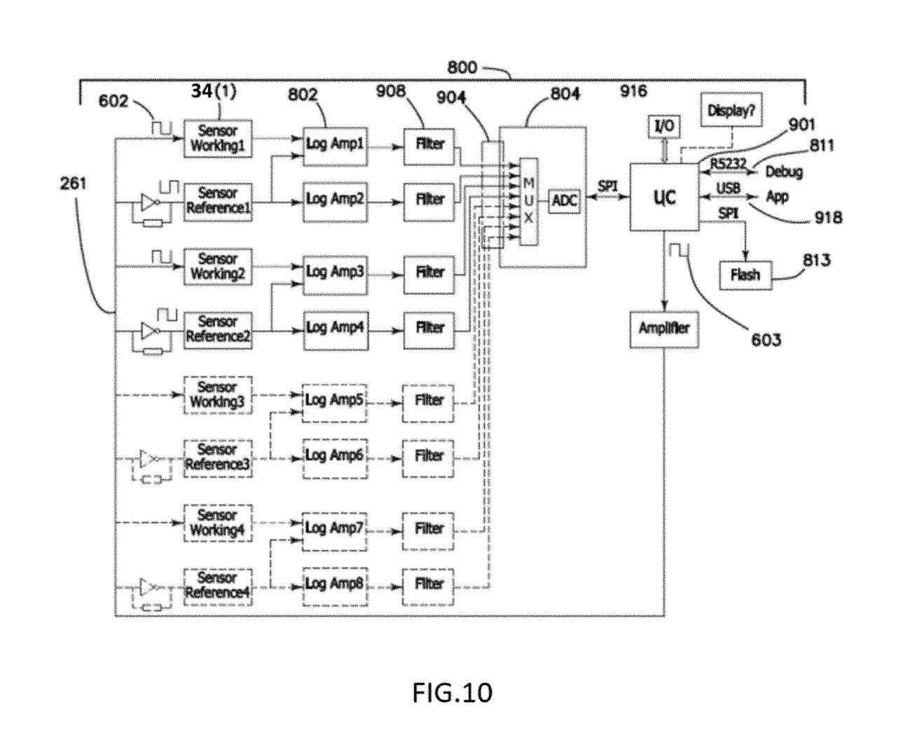

FIG. 10 is one example of a schematic representation of an analog front end (AFE) with selective connectivity to the analog processing platform comprising of an array 261 of sensors 34 connected to a Log Amp 802, filter block 908, MUX 904 with ADC 804 connected to the microcontroller 901 with communication bus SPI 918, RS232 811, flash memory 813 with an exemplary signal output 603. The block diagram depicts a possible architecture of the SAW reader.

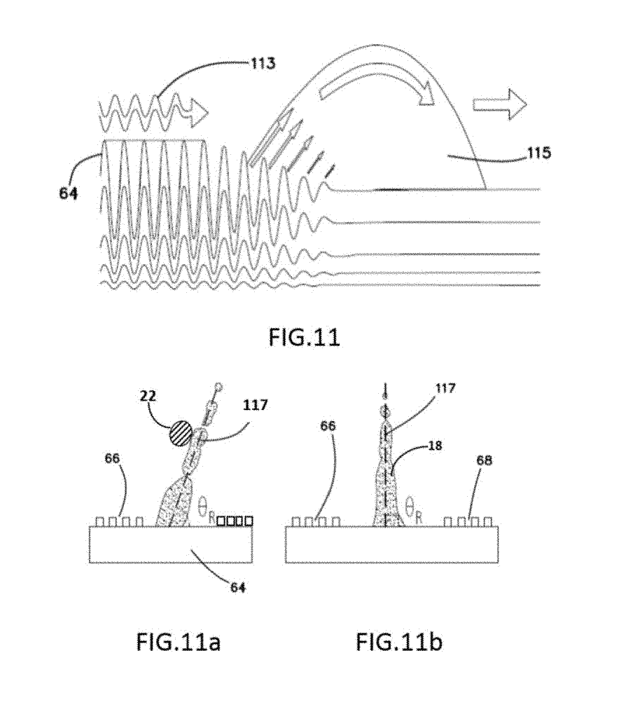

FIGS. 11, 11a and 11b are graphical illustrations of the SAW sensor characteristic electrical phase response relative to mass loading. The simplified representation of the circuit is used to demonstrate the sensitivity of the acoustic sensor as an equivalent RLC circuit, where the load over the circuit is proportional to the energy in the propagation path. The system sensitivity, measured with the added GNP 22 is schematically represented by employing an RLC circuit where the capacitive loading and its equivalent resistive value varies with proportionality to the mass increase on the sensing lane.

In the bulk acoustic wave mode of propagation, the energy disperses from the oscillating surface of the transducer though the coating material. The ratio between the energy dissipated during one period of oscillation and the energy stored in the oscillating system determines the quality factor (Q-factor) of the resonator (The SAW), an important characteristic of the device. In surface, acoustic wave sensors, the energy is trapped near the oscillator surface, which increases the sensitivity of the device in measurements of the surface mass of sensing area 10 in FIG. 1. An acoustic biosensor is a type of a sensor device, where the transducer elements, IDTs 66, 68, are based on a solid slab of piezoelectric material LiTaO₃ 60 that can generate acoustic waves in the sensor's substrate 64. The material used for the acoustic sensor substrate is a quartz crystal cut in specific crystallographic directions (the so-called AT-cut and ST-cut quartz plates) so that the angle 61, of the plate 60, in the quartz crystal supports a shear deformation. The application is directed to two types of piezoelectric resonators, namely, a surface acoustic waves (SAWs) device with a shear horizontal (SH) mode for ST-cut quartz and a Rayleigh surface wave (RSAW) sensor.

FIG. 15c graphically represents the relation between the IDT input 66 and the IDT output 68. The expression describing the circuit is defined by: τ=D/v_{ϕ}+(L−D)/v_{ϕIDTs}. and were v^{ϕ} being the phase velocity in the sensing area and v_{ϕIDTs} is the phase velocity in the IDTs area which remains constant during the sensing event. This corresponds to a phase-shift of 2πfτ. In this way, the condition for oscillation is 2πfτ+ϕₐₘₚ=2mπ, where ϕₐₘₚ is the phase-shift introduced by the RF amplifier. Assuming ϕₐₘₚ remains unchanged, relative changes in phase velocity v_{ϕ}, due to a sensing effect, produces a variation in the time delay τ of the signal through the SAW device. A change in τ modifies the phase condition and therefore oscillation frequency. In this way, relative changes in wave velocity lead to equivalent relative changes in oscillation frequency,

Δf/f₀=Δv_{ϕ}/v_{ϕ0},

where f₀ and v_{ϕ0} are the unperturbed oscillation frequency and wave velocity, respectively, and Δf and Δv_{ϕ} are the shifts in frequency and velocity respectively. Thus, the modification of the phase of delay line due to mass changes can be measured as frequency shifts. Counting the oscillator frequency with a digital frequency counter provides an indirect measurement of the acoustic wave velocities. Generally, the high sensitivity of microacoustic sensors is closely related to the fact that they show a high temperature stability (low TCF) and a large signal-to-noise ratio, which, in turn yields low LOD and a high resolution of the sensor assembly when coupled with a digital circuitry that provides the processing capabilities of the system 46 as shown in FIG. 4.

We can define the impact of the mass loading on SH SAW sensor by characterizing the system 900 in FIG. 5c as a simple oscillator and the mass loading in terms of a delay line. The wave 113 in FIG. 11 propagates when a mass e.g. (gRNA+GNP) spliced onto the DNA site in an aqueous droplet 115, the attenuation and phase shift is represented by inclination of the molecular axis 117. The illustration shown in FIG. 11b indicates similar condition of the wave energy without the attachment of a GNP, but shows that the molecular axis 117 angle is not impacted as the mass threshold is insufficient to affect the measuring apparatus, since it is below the SNR base line of the system's capacity to measure such a value. The embodiments described by FIGS. 1, 2, 3a and 3b overcome such limitations since the system's ability to measure a low concentration analyte (below the minimum LOD detected by the SAW system 900) is augmented with the attachment of a GNP mass to the conjugation site (target DNA 18) employing the CRISPR/Cas9 20 with its additional payload 22 GNP.

Sensitivity Vs. LOD

FIG. 13 is an orthographic representation of a SAW device as a surface acoustic wave sensor with a propagation along its X-axis. The system is configured as having a closed loop architecture. These parameters defining its sensitivity are tailored to the SAWs use to detect bacteria, DNA, or viruses. The LOD depends on the sensitivity of the device and its biological payload. The LOD is directly derived from the ratio between the noise in the measured electrical signal N_{f} and the sensitivity S of the device is a functional ratio of the mass density over the sensing surface, resulting in a phase change over time. For instance, in a closed loop configuration, this noise N_{f} is the root mean square (RMS) value of the frequency measured over a given period in stable and constant conditions. It is measured as signal variation higher than 3×N_{f} the noise level from an effective response variation σᵣ of mass density over linear surface area. Based on this parameter, it follows that the minimal effective signal above the SNR value relative to the LOD is given by the expression:

$$LOD = \frac{3 \times {N}_{f}}{{S}_{σ}^{φ} \times {φ}_{0}} {Δσ}_{r} = \frac{3 \times {N}_{f}}{{S}_{σ}^{φ} \times {φ}_{0}}$$

where fₒ is the operating frequency, S_{σ}^{ϕ} is the phase (or gravimetric) sensitivity which is a term describing the relationship of the phase shift and the added mass relative to the signal fidelity as defined by the expression, and ϕ₀ is the unperturbed phase. In this way, we can also define the LOD for phase measurements as:

$$LOD = \frac{3 \times {N}_{φ}}{{S}_{σ}^{φ} \times {φ}_{0}} {Δσ}_{r} = \frac{3 \times {N}_{φ}}{{S}_{σ}^{φ} \times {φ}_{0}}$$

where N_{ϕ} is the noise in the measured signal above the 3×N_{f} of the noise-floor and it is represented as the mass add on to the sensing area and indicated by the reader as a differential output in terms of phase shift.

SAW Biosensor Structure

FIGS. 4a and 4b is a graphic representation of the SAW biosensor constituent (elements) modeled as shown in FIG. 4b and schematically comprising of three variables: coating layer with shear modulus, μ, the layer density p and viscosity of the medium η. while the remaining subscripts terms in the figure represented as S, L, SA, C and F denotes the substrate, guiding layer, sensing area coating and fluid layers, respectively. FIG. 4a graphically illustrates the model defined by FIG. 4b where the transducer is operated when the difference between the mechanical properties of the piezoelectric substrate 60 and the guiding layer 62 generates a confinement of acoustic energy in the guiding layer 62, thereby slowing down the wave propagation velocity, but maintaining the propagation loss. In particular, the condition for the existence of a Love wave mode is that the shear velocity generated by the LiTaO₃ crystal 60, relative to the guiding layer 62 material (SiO₂) is such that the wave velocity of the guiding layer (v_{L}=(μ_{L}/μ_{L})^{1/2}) is less than that of the substrate layer velocity (vₛ=(μₛ/ρₛ)^{1/2}), the sensitivity of the sensor is increased, where μₛ<μ_{L}. A large value of that ratio (higher μₛ and lower μ_{L}) leads to a stronger entrapment of the acoustic energy and thus greater sensitivity. Hence, the benefit of the guiding layer is an enhanced sensitivity due to reduction of the insertion loss, thereby the phase shift above the system noise level, is represented accurately as the mass deposition and its measurement. The model shown by FIG. 4b serves as the basis for the applicant in providing the means by which we can improved the LOD.

SAW Substrate Coupling (K²)

In one embodiment, the application demonstrates that modeling the effect of the guiding layer for the Love modes is a parameter which influences the substrate coupling factor K². Increasing electromechanical coupling coefficient (K²) improves the sensitivity of the device. In addition, it influences the temperature behavior, since it modifies the temperature coefficient, in relation to the materials used for guiding layer, those materials with a low shear velocity, low acoustic loss and low insertion loss seem to be the optimized for developing sensitive biosensors as it is used by this application. Materials, such as polymers, silicon dioxide (SiO₂), gold (Au) and zinc oxide (ZnO) have been used as guiding layers, and due to the wide frequency band availability of these materials, the issue of signal loss becomes negligible. However, in a certain frequency band, the scattered waves are in phase, adding them constructively and causing a very strong reflection which distorts the transducer frequency response.

In one embodiment of the proposed system, the modeling noted by FIG. 4a indicate that the IDTs 66 and 68 formed under the guideline of an ideal resonator geometry, reduces the unwonted Bragg reflection which occurs when the phase matching condition 2 p_{g}=nλ is met, where p_{g} is the grating periodicity and n the mode number. For a single-electrode IDT (see FIG. 5b), this condition occurs at the resonance condition λ=p. Thus, double-electrode (or double finger pair or split-electrode) IDTs of FIG. 5a are used to avoid this unwanted effect. In double-electrode IDTs there are four strips per period and thus, the Bragg reflection, which is the effect of the constructive or destructive interference intensifies because of the cumulative effect of reflection, can be suppressed at the SAW resonance frequency.

SAW Crystal Orientation

This application employs the SH SAW sensor, where the crystal is based on a 36° rotated Y Cut and X-axis propagating wave (LiTaO₃) (36° YX LTO) direction (as shown by FIG. 13) as the piezoelectric substrates 60, in which SAWs is generated and detected using conventional lithographic metal deposition fabrication to form the interdigital transducers (IDTs) 66,68. As is known, IDTs excite both the surface skimming bulk acoustic waves with SH-polarization and the leaky SH SAW. The phase velocities of these two types of waves on the free substrate surface are almost equal (a difference being on the order of 10⁻) and the SAWs are effectively converted into volume waves. To inhibit this conversion, a conducting film that “presses” the SAW to the substrate surface and it is coated over the region between IDTs. In some cases, the wave energy concentration at the surface (and, hence, the sensitivity) is increased by applying a several micron-thick dielectric films or e.g., SiO₂, possessing waveguide properties with respect to SH SAW. Thus, the sensor structures—36° YX LTO,—substrates contains surface regions with different electrical and acoustical properties as shown by the modeling description of FIG. 4b.

As noted by the prior art, the SAW in the Love mode operation is a complex of variables (see modeling description of FIG. 4b) where the SH polarized acoustic waves are very effectively reflected by the substrate edges and various inhomogeneities are present on the surface, (a noise generating sources that this application take into considerations by applying magneto-optical analysis—(Snell's Law and Bragg reflection), to reduce the artifact of wave reflective and refractive responses due to geometry and other parameters noted above, as the wave travels through the guiding layer. Sensors that operate in a continuous mode, used in most experiments, generate reflections, which lead to distortions in the amplitude and phase characteristics of the sensors. To ensure a correct measurement of the system response (which usually represents a change in the signal amplitude and/or phase), it is necessary to take special measures either to decrease these distortions or eliminate them, e.g., by using SAW excitation in a pulsed mode or by tailoring the microfluidic chamber geometry with its guiding layer through the analytical use of magneto-optical optimization and the use of the material properties to reduce such artifact effects.

SAW Limit of Detection (LOD)

The critical parameters which affect the SAW sensitivity and affect the minimalization of the LOD are:
Minimal mass loading above the SNR measure,

$$( LOD = {Δσ}_{r} = \frac{3 \times {N}_{f}}{{S}_{σ}^{φ} \times {φ}_{0}} ) .$$

The conjugation method of isothermal DNA editing employing CRISPR-Cas9 with its added mass of GNP to reduce the LOD.
The employing of down conversion circuit to enable a time domain suitable for signal capture of biological kinetics.
Microfluidic integrated sensor to control hydrostatic flow and capture-rate of hybridization statistics.
The use of GNP conjugated particles as markers detectable by use of a electro-acoustic wave.

The transducing area includes the interdigital transducers (IDTs) 66, 68, which are metal electrodes, sandwiched between the piezoelectric substrate 60 and the guiding layer 62. A typical IDT pattern is diagrammatically depicted in FIG. 4a. The input IDT 66 is excited electrically by applying an RF signal from RF source 97 which launches a mechanical acoustic wave into the piezoelectric material 66 as graphically depicted and described by FIG. 11.

In one of the preferred embodiment of the proposed sensor we employ the model illustrated in FIG. 4a, providing a tool that calculates the radiation conductance G, the acoustic susceptance Y, (the inverse of impedance Z) and the frequency response for the system, to optimize the system sensitivity thereby reducing the LOD. The model includes optimization for the aperture height. The effects of triple transit echoes (Bragg reflection), have been added to the model from the Impulse response, where one can calculate the wavelength (λ) and the number of finger pairs (Np) 66,68, using the following equations:

$$λ = \frac{v}{fo} \frac{v}{fo} ,$$

where V is the acoustic velocity in the media, and fofo is the center or synchronous frequency. The frequency (e.g. 300-400 MHz) is an indication of where “sweet spot” tuning occurs and the center frequency optimal design must match the IDT resistance (real impedance) to the source resistance.

The device aperture is adjusted so that the IDT design achieves the correct resistance. Where the wave energy is guided through the guiding layer (waveguide) 62 up to the output IDT 68, where it is transformed back into a measurable electrical signal (phase shift) 46. The sensing area 64 is the area of the sensor surface, located between the input IDT 66 and output IDT 68, which area 64 is exposed to the analyte. A simplified diagram of a SAW device is depicted in FIG. 5b, where FIGS. 4, 4a, 5a and 5c are further elaborations of the sensor.

The sensor uses shear horizontal (SH) surface acoustic waves, which are frequently used for liquid-loaded biosensing applications. In SH-SAWs, the particle displacement is in the plane of the surface. SH-SAWs are not affected or damped by liquid loading, as compared to Rayleigh waves. On the other hand, almost all SH wave propagation on various substrates results in leaky waves which also leak into longitudinal and shear vertical wave components when excited. For this reason, special cuts of typical wafer types of wafers are typically used for SH waves, in which the energy is highly concentrated on the SH mode. Typical wafer types used in this application employ a SH-SAW with ST cut quartz, (36° Y-cut of lithium tantalate (LiTaO₃)).

The sensing mechanism of SH-SAW sensors relies on the change of SAW speed either by change in mass loading (most biological and chemical sensors) or by changing physical parameters, such as the sensor native frequency, mode of detection e.g. phase shift or amplitude change, geometry layout of the IDT's, or the delay dielectric material forming the waveguide.

Phase Shift Vs Mass Loading

In one embodiment, the reader 800 employ (in a background mode) the Sauerbrey equation, correlating changes in the oscillation frequency of a piezoelectric crystal with the mass deposited on it, for computational modelling of the SAW as a resonant cavity when the crystal is perturbed due to resonance upon application of layers at the sensor surface. As illustrated by the equation below, the expression correlates the changes in oscillation frequency (of a piezoelectric crystal), with the mass deposited on its sensing lanes and provides an added value to the system reliability as it enables a reference theoretic point of comparison to the proposed system, where kinetic of hybridization is commuted.

Further illustration of the embodiment is the co-generation of theoretical plot to assess the performance of the SAW devices in practice and in accordance with the modeling noted by FIG. 4b, the system measure a Δϕ (phase change), not Δf frequency change, but one can be deduced from the other by the changes in acoustic wave velocity, Δv, upon layer addition, (see modeling details noted in FIG. 4b). The perturbation that causes a certain amount of Δv has been determined theoretically for some ideal materials, considering the application of a linear elastic mass defined as:

$$\frac{Δ f}{f} = \frac{Δ v}{v} = {ζρ}^{′} hf$$

where ρ′ is the added mass density and h is the height of the added mass, and the perturbative shift in frequency f is proportional to ζ (which depends on device parameters and can be experimentally verified). Here we define the resonant mode as the mode that results in maximum average displacement over time. For any linear elastic mass addition, we expect a change in frequency to be proportional to the change in added mass based on the parameters noted by our model.

In this application, we consider the process of minimizing the added mass to meet the minimal LOD's threshold for which the SAW resonator can display a measurable signal above the noise floor of the system, i.e. its native SNR, as we deploy a method of DNA editing whereby the specific area of the gene is modified via RNA guide (gRNA) so that the locus of interest on the code specific area is modified with the added mass of GNP. The process of such editing and splicing the specific gene with its conjugated mass of GNP is described in FIGS. 1, 2, 3a and 3b. The resultant application of the gene splicing and conjugation is the substantial reduction of the numbers of analyte units per sample and therefor the minimization of the LOD to a few DNA strands.

SAW Array Configuration

FIGS. 6, 7 and 8 are schematics of a plurality of the SAW cell arrays 261, where the individual SAW units and their corresponding source follower amplifiers 119, are configured to enable multiple analytes to be processed simultaneously. The array configuration and its geometrical layout is a function of its use; the cells 34 in an array can be arranged in an arbitrary number of dimensions and geometrical configurations, such as a square, hexagonal, or any other spatially arrangement. Topologically, the SAW cells 34 can be arranged on an infinite plane or on a toroidal space.

Microfluidic and Convection Enhanced Delivery

The microfluidic chamber assembly 263, indicated by a phantom line in FIG. 4, is integrated with the SAW sensor 34 and may assume a variety of hydodynamic topologies to improve fluid flow and obstruction avoidance due to sedimentation of proteins on chamber' surfaces, as well as to provide an optimal axial-flow of the analyte with its buffer onto the sensing and reference lane 206 and 207 respectively, further preventing a Gaussian distribution and hybridization on the nearest or adjacent individual lane due to transverse flow over the sensing lanes. All the sensing and reference lanes are commonly located on the sensing area of the device, the lanes are metrically similar and their functionalization prior to use are such that a template separates the reference lane from the sensing lanes to load the non-specific antibody onto the reference lane and the specific antibody in question placed on the active sensing lane. These preparatory processes conduct at the manufacturer level and are stable for long periods of time with refrigeration at 4° C.

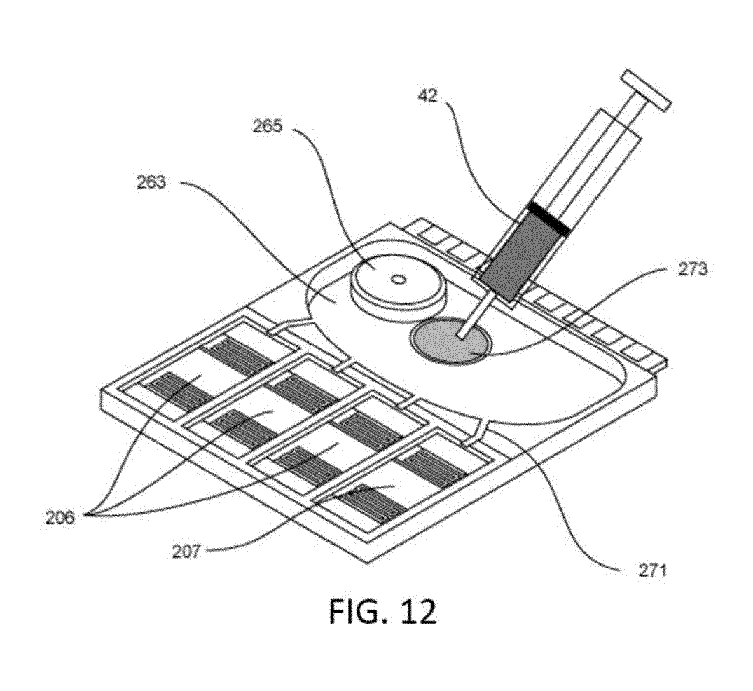

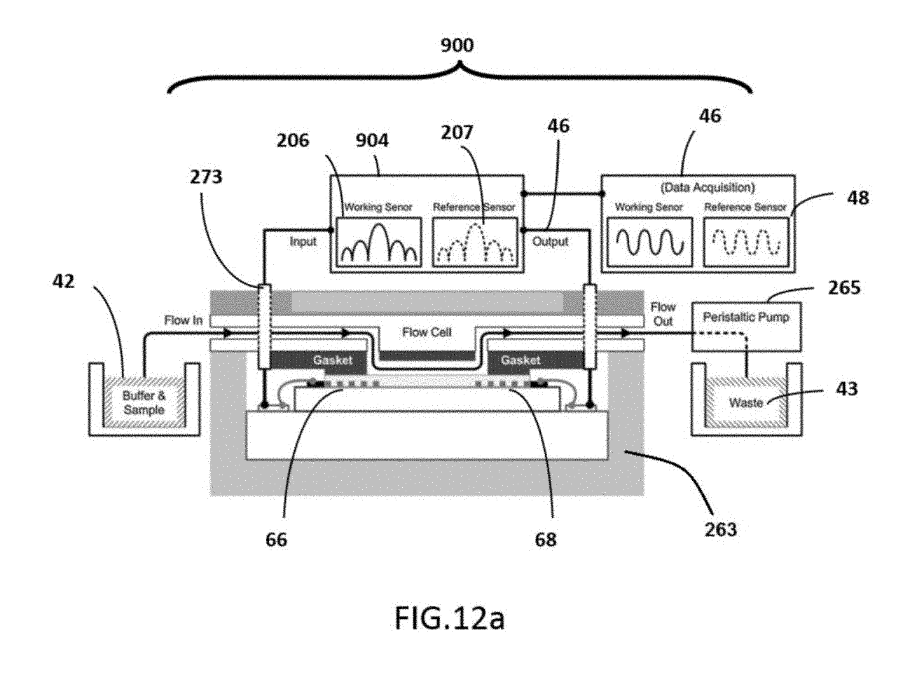

FIG. 12a depicts the microfluidic platform 263, integrates with a SAW sensor 34, a regulated pressure 265, peristaltic pump 265 and a programmable electromechanical valve 269 (not shown for clarity). This embodiment provides for accurate control over the displacement of liquids introduced within the system via septa port 273 by simultaneously pressurizing multiple outlet ports 271 of the microfluidic device in contrast to systems where liquids are solely driven by centrifugal and capillary forces. The method adds a new degree of freedom for fluidic manipulation, which represents an improved control of hydrodynamics, thereby reducing the error associated with directly pipetting into the sensing and reference lanes 206,207, where gravity is insufficient to overcome the capillary forces exerted on biological and inorganic constituents forming the analyte. The microfluidic chamber 263 indicated by a phantom line in FIG. 4, contains the following features, which enable the flow of analyte and buffer in an aqueous form through the surface fluid channel inlet and the fluid channel outlet, and which passes through the device active area 36. The microfluidic chamber is hermetically sealed by an O-ring 69. Within the sealed chamber is the sensing area containing the SAW sensing lane 206 and reference lane of the SAW cell 207.

SAW Reader

FIG. 7 is a schematic block diagram of the SAW array 261 of the SAW cell 34, with its analog front end (AFE) 904, comprising (for simplicity) log amp 802, filter 803, MUX 905, ADC 804, and amplifier 119. Sensor cell 34(s) are configured in a fashion which enable a common power source and where the array 261 can be processed and analyzed by the electronic reader 900 with a display unit 716. A man familiar with the art of electronics can conceive of adding a wireless communication link as well as data transmission to the cloud for storage and analysis.

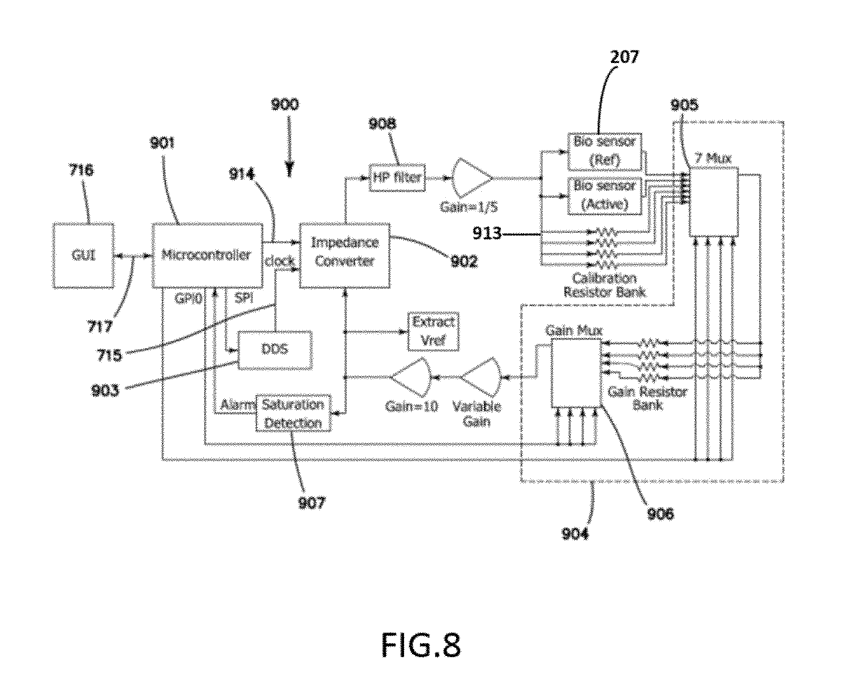

FIG. 8 is a schematic representation of the SAW electronic interface or reader 900 comprising a GUI 716, communications bus RS-232 USB or SPI 717, directed by microcontroller 901, clock 715, HP filter 908, and other components and detail associated with the analog front end as is well known in the art. The schematic depicts a specialized feature such as shown by reference designator, describing the calibration resistor bank 913 with its complementary gain mux multiplier 906, this circuit detail, enable the analog front end 904 to perform an accurate and representative scaling operation-(guided by an instruction-set and look-up tables embedded in microcontroller 901),—associated with the actual concentration value of the analyte and act as a trigger for adjusting gain multiplexer to mimic the rate of hybridization kinetics data, generated by the SAW cell units 34.

The apparatus 904 of FIGS. 7 and 8 further describe how the system 900 acquire and multiplexed the data, perform analysis of the sensed biological process by measuring and recording the hybridization dynamically. The system 900 further act as a flow cytometric analysis of analyte-antibodies and/or DNA detection while employing the Crisper/Cas 9 system for editing specific gene within the DNA code.

FIGS. 7 and 8 further enable the system 900 to perform detection and analysis of multiple variables simultaneously. This approach of using the Crisper Cas9 for gene editing method (described in FIG. 1) can be extended in an architecture which performs simultaneous measurement of multiple different analytes. The system consists of an array of SAW cells 261 with a distinct set of specific probes and the resultant output of the hybridization are addressable by the resident microcontroller 901 interfaced with a digital signal processing on board and with suitable software routines.

In one embodiment, we employ individual sets of microspheres such as gold nanoparticles (GNPs) 22 that can be modified with reactive components such as antigens, antibodies, or oligonucleotides, and/or guided RNA 20 and then mixed to form a multiplexed assay set as shown in FIG. 1, where the GNP 22 and the gRNA 20 form a conjugate. The microfluidic chamber 263 controls the flow as a cytometer and the apparatus 900 performs a near real-time data processing, allowing multiple independent reactions to be analysed simultaneously. The system 900 of FIG. 8 performs qualitative and quantitative immunoassays for multiple serum proteins while using an array 261. The system can be used to perform DNA sequence analysis by multiplexed competitive hybridization with different sequence-specific oligonucleotide probes.

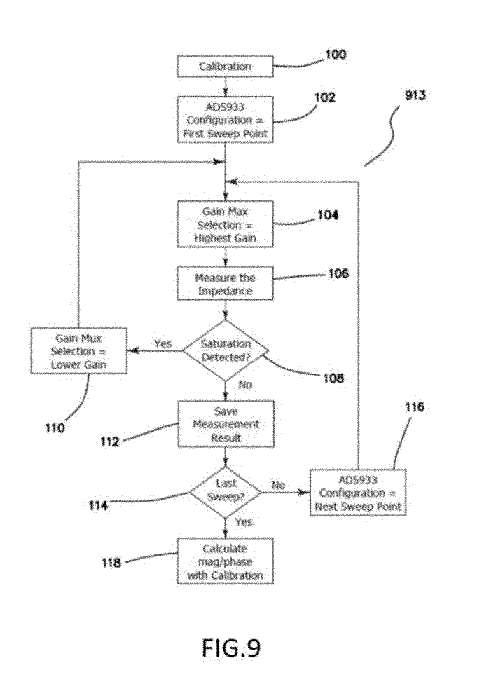

Further elaboration of the system 900 operation is noted by following FIG. 8 signal flow, where microcontroller 901 (such as PIC32MX380F512L) is used to direct traffic, CPU (MC) 901 further fetches instructions, decodes each instruction, fetches source operands, executes each instruction and writes the results of instruction execution to the proper destinations. The microcontroller 901 selects via gain multiplexer 906 a cell and compares the differential outputs of the SAW sensors 34 (or array 261) with values in look-up table suitable for the application. An example of such is the use of AD5933, which is a high precision impedance converter 902 that combines an on-board frequency generator with a 12-bit, 1 MSPS, analog-to-digital converter (ADC). The frequency generator in impedance converter 902 allows an external complex impedance to be excited with a known frequency. The response signal from the SAW cell or biosensor is sampled by the on-board ADC in impedance converter 902 and a discrete Fourier transform (DFT) is processed by an on-board Direct Digital Synthesizer (DDS). A detail scheme of the ADC 804 is shown in FIGS. 15a and 15b. The DFT algorithm returns a real (R) and imaginary (I) data-word at each output frequency. Once calibrated signal is achieved by comparing between biosensor signal cell and biosensor reference cell, the magnitude of the impedance and relative phase of the impedance at each frequency point along the sweep is easily calculated by the resident math-coprocessor on board the MC 901. The DDS 903 unit (such as AD9834) defines the clock traffic within the apparatus 900 with its other functional blocks of the analog front end 904. The SAW sensor outputs is constantly compared by the saturation detector 907 and enables a selection of the appropriate gain necessary for linearization as shown and described by the flow diagram of FIG. 9. Employing direct digital synthesizer (DDS) 903, where the signal undergoes a multi-gain stage which increases the impedance range, and where saturation detection circuit determines the appropriate value to be selected from the gain bank resistor coupled to the input of gain mux 906.

Reader Embedded Instructions

FIG. 9 is a flowchart detailing the auto-gain selection software logic in microcontroller 901 designed to select the proper post-amplifier gain based on the output of saturation detection circuit 907 to insure the phase shift signal within impedance converter 902 is within its linear range. In one embodiment, the embedded software is designed to function as a state machine to control the measurement sequence of converter 902 over output 46.

FIG. 9 further illustrates the methodology of the measurement sequence of circuit detail defining the converter 902. Calibration begins at step 100 followed by configuration of the circuit 902 at the first frequency sweep point at step 102. The gain of gain multiplexer 906 is set at its highest gain at step 104. The phase shift at the first sweep is then measured at step 106. A determination is made at step 108 whether or not saturation has been achieved. If saturation has been achieved, then the gain of gain multiplexer 906 is set to a lower level at step 110 and the process returns to step 104 for the next series of sweeps. If saturation has not been achieved, then a measurement result is saved by microprocessor 901 at step 112. A determination is then made at step 114 whether or not the frequency sweep just made is the last one to be made in the series or not. If not, then the next frequency sweep point is selected at step 116 and the process returns to step 104. If the frequency sweep made is the last one of the programmed series, then the calibrated magnitude and phase of the impedance is calculated at step 118.

The circuit architecture shown in FIG. 10 includes the analog front end 904 coupled between the sensor(s) 34 in an array 261 of a plurality of sensors 34 and reference cell pairs 34(n), each pair coupled to a corresponding log amp 802 and filter 908 and through multiplexer 804 to the digital back end including microprocessor 901. The output signal from filter 908 is a continuous analog signal. The apparatus mimics the underlying biological processes employing discrete state spaces. The data stream is then manipulated by the microcontroller 901 which arithmetically describes the physical process operating as time-varying quantities. The analog front end 904 and the digital peripherals shown in the figure, record, store and analyze the hybridization as well as the diffusion processes, which underlay the biology investigated by the apparatus 900.

Impedance and Phase Shift

The SH-SAW is excited on a 36° YX LiTaO₃ and the right-angle edge of the substrate is used to reflect the SAW. The SAW has two components of particle displacement (see FIG. 13). One is parallel to the surface along the direction of the wave propagation 56, and the other is normal to the surface 57. The desire to sense the liquid phase using a SAW device is complicated by the excessive energy losses experienced at a solid and liquid interface. Displacements normal to the surface generate compression waves, which dissipate the wave energy in the liquid. Therefore, liquid phase sensing using the SAW device is difficult and therefore, we employ shear horizontal mode SAW (SH-SAW) which are not affected by this energy loss mechanism.

The magnitude of the output signal is the function of the ratio of the signal's wavelength 113 and the distance 2d 2d, 114 in FIG. 13. The sinusoidal electrical input signal generates an alternating polarity between the fingers of the interdigitated transducer. Between two adjacent sets of fingers, polarity of IDE fingers is switched based on polarization e.g. (−+−+). Thus, the direction of the electric field between two fingers will alternate between adjacent sets of fingers. This creates alternating regions of tensile and compressive strain between fingers of the electrode by the piezoelectric effect, producing a mechanical wave at the surface. As fingers on the input IDT's of the sensor, where it experiences the same level of compression or tension, the space between them—known as the pitch—is the wavelength λ of the mechanical wave. The synchronous frequency f₀ of the device with phase velocity vₚ and pitch p is defined by the expression:

$${f}_{0} = \frac{{v}_{p}}{p} {f}_{0} = \frac{{v}_{p}}{p}$$

Defining the pitch and the electrodes length deposited over the crystal LiTaO₃ is a critical parameter in establishing the resolution of the sensor. Hence, the limit of detection (LOD) is directly related to the frequency domain that operates the device and it is linearly related to the frequency, the higher the frequency the higher is the resolution.

If the distance 2d is equal to the wavelength λ, the magnitude of the output voltage is maximal. The corresponding frequency is called the “center” or the synchronous frequency of the apparatus 900. The magnitude of the output voltage decays as the frequency shifts from the center frequency. It means basically, a SAW device is a transversal bandpass filter, see layout on FIG. 13 and X-axis propagation of wave-vectors 56, and its transversal waves output 57 in FIG. 12, which in the proposed configuration is being altered by the added mass of GNP, whereby the device electronic scheme is attenuated by the added mass and is represented as a proportional phase shift. In transversal like filters, the phase characteristic is a function of the distances between the electrodes and the amplitude characteristic is a function of the number of electrodes and their lengths. The IDT geometry is capable of almost endless variation, leading to a wide variety of devices. If the electrodes are uniformly spaced, the phase characteristic is a linear function of frequency, e.g., the phase delay is constant in the appropriate frequency range.

FIG. 14 is understood within the context of FIGS. 5a, 5b and 5c where the SAW device is then called a delay line as diagrammatically depicted in 5a and where a reference lane 207 (functionalized with non-specific antibody) and sample lane 206 (functionalized with specific antibody) generate a differential output 46. Input IDT 66 is coupled to an amplifier 119 (The analog front end 904 is omitted for simplicity). The phase shifter outputs from reference lane 207 and sample lane 206 (with its analyte specific biological probe or antibody) are coupled to a mixer 123, whose output is coupled through low pass filter, thereby enabling the electronic apparatus 900 to act as a frequency counter, resulting in a phase shift value proportional to the mass accumulation over the time. The technique of employing a mass loading over the sensing and reference lanes is fully described by the above Figures.

Top Level Architecture of SAW Platform

FIG. 15 is a top-level architecture of the system comprising a RF synthesizer 602, an RS232 communication link 811, local oscillator (LO) synthesizer with voltage control oscillator (VCO) 903, a SH-SAW cell 34, analog front end 904, analog to digital converter (ADC) 804, and field-programmable gate array, (such as Xilinx FPGA), 890, as part of an electronic scheme where the SAW output signal is enabled with a lower temporal widow which improves sampling rate and reduce jitter and noise, while preserving the phase and amplitude of the native signal.

FIG. 15A is a block diagram depicting a phase measurement system employing digital IIQ Demodulator 980, demodulating, and setting up a clock cycle to enable processing and analysis of the native signal in a time domain-scale by employing a Mixer/LPF 803 and by down-converting the RF signal from 325 MHz to an IF signal set to 500 KHz. The scheme of employing the Digital I/Q Demodulator and LO synthesizer enables the system to capture data at a rate of microseconds as opposed to picosecond domain. This electronic transaction conserves the basic properties of the native system such as amplitude/phase.

In one embodiment, the system's use of a lower frequency IF signal relaxes an ADC sampling rate and clock jitter requirement. A common local oscillator and a differential phase calculation eliminates IF synthesizer jitter. Digital I/Q demodulator 980 produces an accurate phase calculation due to perfect 90° phase shift.

FIG. 15B is a further elaboration of the block diagram noted in 15A, depicting the functional blocks forming the technique of down-conversion from MHz to KHz scale. A mixer with Local Oscillator/Synthesizer with Voltage-Controlled Oscillator, the signal undergoes a LPF 903 while down-converting the RF signal at 325 MHz from RF source 602 to an intermediate frequency (IF) 975 @(250 KHz) with phase information passing through (phase change conserved), shifting the RF to a lower frequency (IF), filtering out unwanted high frequency components 803 (local oscillator+RF), using amplifier 119 to adjust the signal suitable for ADC's 804 dynamic range with common local oscillator and differential phase channels eliminating synthesizer phase noise (jitter) 980.

A clear representation of the mixer's 903 performance is shown when the system is algebraically analyzed: the mixer produces two side bands,

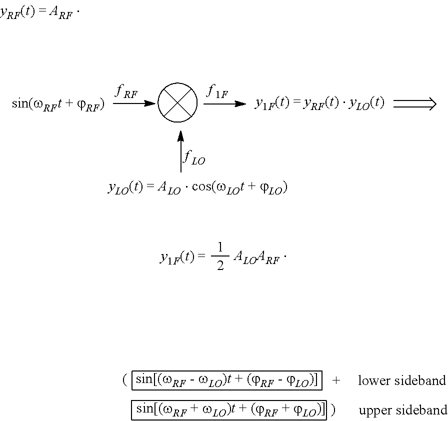

Down-conversion: after low pass filtering the upper sideband

$$⇒ {y}_{IP} ( t ) = {A}_{IP} \cdot sin ( {ω}_{IP} t + {ϕ}_{IP} ) \\ {ω}_{IP} = {ω}_{RF} - {ω}_{LO} \\ \begin{matrix}{A}_{IP} = \frac{1}{2} {A}_{LO} {A}_{RF} ∼ {A}_{RF} with constant {A}_{LO} \\ {ϕ}_{IP} = {ϕ}_{RF} - {ϕ}_{LO} ∼ {ϕ}_{RF} with constant {ϕ}_{LO}\end{matrix} \}$$

Phase and amplitude of the RF signal are conserved during down conversion, but time period corresponding to the phase value is dramatically enlarged. This feature of the electronic scheme enables the system 900 to perform a sampling and analysis of the acquired signal in a time domain that improves data collection by order of magnitude and thereby, reduces the LOD minimum required to obtain similar resolution which otherwise will necessitate higher concentration of the analyte in question.

FIG. 15c is a graph of LT-Spice simulation where the original phase values before down-conversion and where the reference signal is set at 325 MHz, resulting with a delta phase equal to 90° and time sampling interval is equal to 0.77 ns.

FIG. 15d is LT-Spice simulation demonstrates a result that the electronic scheme of down-conversion from higher frequency (325 MHz down to the intermediate frequency of 250 KHz) is conducted, resulting with delta phase equal to 900 and time is equal to 1 μs.

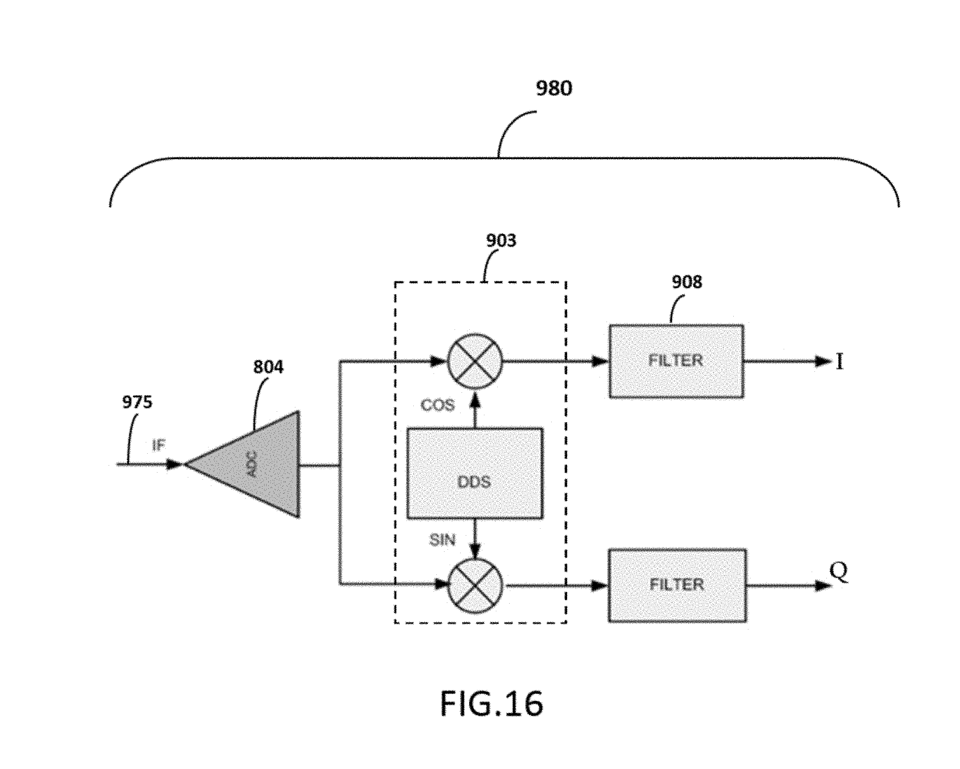

FIG. 16 is a visual representation of digital I/Q demodulator 980. Digital I/Q demodulator is implemented in a field programmable gate array (FPGA) 890, mixer 903 and LPF 803 implemented in digital domain, to produce an accurate phase calculation due to accurate 90° phase shift by DDS (Direct Digital Synthesizer) 903 to minimize the phase misalignment. The operation of an IQ-demodulator 980 can be explained by representing its RF input signal S_{RF}(t) as a combination of two double sidebands modulated quadrature carriers: the in-phase component I(t) and quadrature component Q(t) are baseband signals that can be viewed as inputs to an ideal IQ-modulator generating S_{RF}(t). An I/Q-demodulator achieves perfect reconstruction of I(t) and Q(t) by exploiting the quadrature phase relation between S_{I}(t) and S_{Q}(t). The frequency-domain representation of a −90° phase shift corresponds to multiplication by the Hilbert transform which converts a spectrum with even symmetry around ω=0 (hence preserving the amplitude and phase components of the native signal), to a spectrum with odd symmetry and vice versa. The spectra of S_{I}(t) and S_{Q}(t) therefore exhibit different symmetries; S_{I}(t) has even symmetry, S_{Q}(t) has odd symmetry. Down conversion of the even RF input component S_{I}(t) with the even local oscillator (cosine) retrieves I(t), while S_{Q}(t) with the odd local oscillator (sine) retrieves Q(t). Cross-combinations of even and odd yield zero. An error cp on the quadrature relation between the local oscillator outputs causes crosstalk between the I- and Q-channels. Using the I-phase channel as reference, an even component is introduced in the Q-channel LO, resulting in a contribution of I(t) to the Q-channel output Qₒᵤₜ(t).

GNP as Single-Electron Transistor

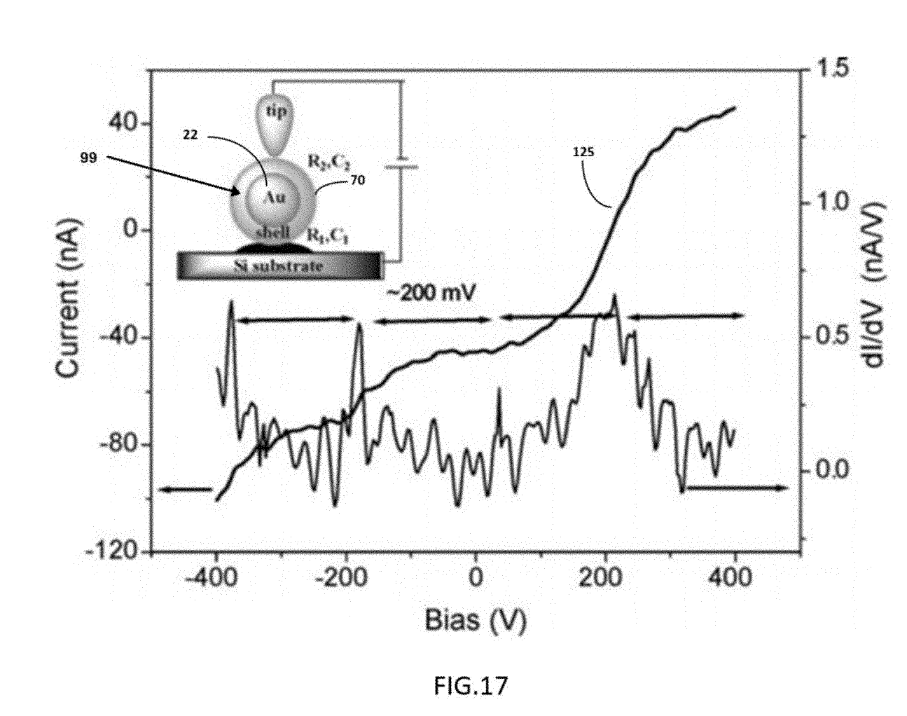

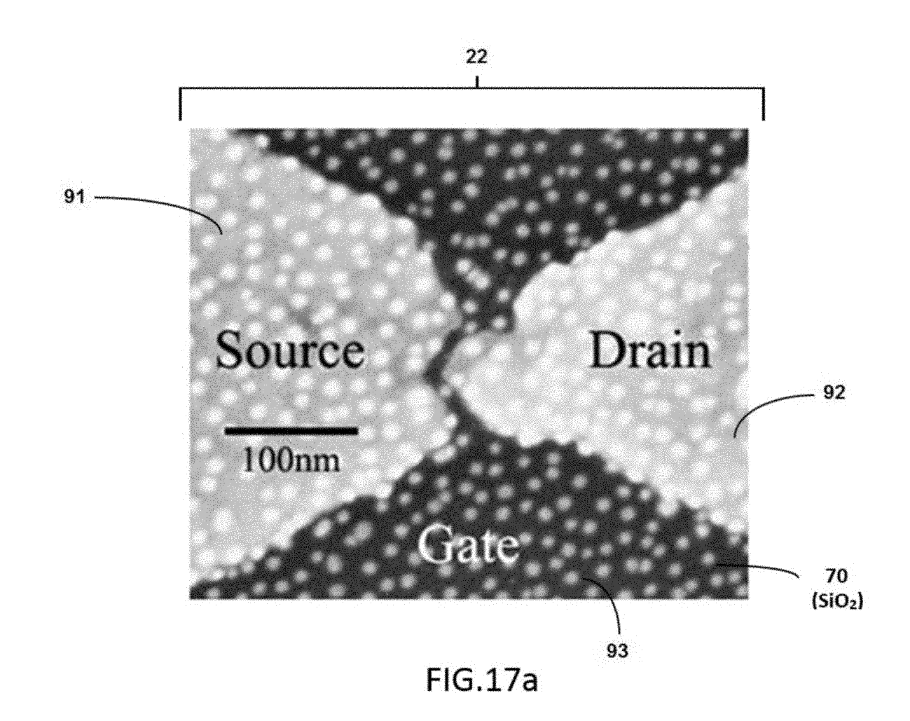

FIG. 17 is an orthographic depiction of gold nanoparticles 22 coated with SiO₂ shell 70, fabricated into two-dimensional array on a silicon surface by an AFM tip-confined guiding template technique, to form a two-dimensional single electron transistor (SET) 99 device. The assembled array of Au—SiO₂ composite nanoparticles exhibits a well-pronounced Coulomb staircase whose period is 200 mV at room temperature, which is a promising candidate for future room-temperature single electron transistors. The current/voltage (I-V) and dl/dV vs V_{sd} curves 125 are graphed in FIG. 17 for one Au—SiO₂ composite nanoparticle confined on the silicon surface.

FIG. 17a is an example of chemically synthesized gold nanoparticles assembled to form a single electron transistor. The principle of operation of a SET is based on Coulomb blockade in a tunnel junction. The tunnel junction is, in its simplest form, a thin insulating barrier such as SiO₂ forming the gate 93 between the two conducting electrodes. Vishva Ray et al reported in Nature Nanotechnology 3, 603-608 (2008) on the development of a CMOS-compatible fabrication of room-temperature single-electron devices operating at room temperature. This was made possible using CMOS fabrication technology and implementing self-alignment of the source 91 and drain 92 electrodes, which are vertically separated by thin dielectric films. Experiments demonstrate clear Coulomb staircase/blockade and Coulomb oscillations at room temperature. In one embodiment, we teach the use of a GNP, chemically modified to form a beacon which responds to ultrasonic waves rather than electrical currents as the excitation mechanism (see Daniel R. Koenig et al in-Nature Nanotechnology 3, 482-485 (2008) titled “Ultrasonically driven nanomechanical single-electron shuttle”).

This application, although not claiming novelty in single electron transistor (SET) topology, rather indicates the possibility of employing the GNP as a mass enhancement as described in FIGS. 1, 2, 3a and 3b, for reducing the LOD and thereby improving the limit of detection the apparatus 900 is capable to measure. Further use of the GNP as a “beacon” responding to acousto-electric radiation generated by the SAW device is contemplated by this application as a method by which a site-specific of a gene on DNA strand can be identified by the conjugating technique enabled by the CRISPR-Cas9 methodology.

In one embodiment, this application claims the use of mass-enhancement modality of the GNP with additional properties associated with the use of a chemically-modified GNP to form a transistor-like behavior, as it is defined by its Coulomb blockade geometry.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments include other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. It is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

1. A method of reducing the limit of detection in a surface acoustic wave sensor (SAW) comprising:
attaching a plurality of DNA segments to a detection surface of a SAW;
performing a CRISPR/Cas9 preparation of the DNA segments to bind a selected protein into at least one of a plurality of the DNA segments;
conjugating a nanoparticle to the selected protein; and
measuring the number of DNA segments with conjugated nanoparticles using the surface acoustic wave sensor (SAW).

2. The method of claim 1 where conjugating a nanoparticle to the selected protein comprises conjugating a gold nanoparticle to the selected protein.

3. The method of claim 1 where performing a CRISPR/Cas9 preparation of the DNA segments to bind a selected protein into a selected target site on at least one of a plurality of the DNA segments comprises:
functionalizing the detection surface of the SAW with streptavidin;
binding a first Cas9 protein with a first guide RNA to a first selected target site on at least one of the plurality of DNA segments, the first Cas9 protein and the first guide RNA comprising a first RNA/Cas9 pair;
biotinylating the Cas9 protein;
binding the biotinylated Cas9 protein to the streptavidin; and
binding the selected protein as a second Cas9 protein with a second guide RNA to the second selected target site on at least one of the plurality of DNA segments, the second Cas9 protein and the second guide RNA comprising a second RNA/Cas9 pair.

4. The method of claim 1 further comprising providing a semiconductive structure on or in the nanoparticle, and wherein measuring the number of DNA segments with conjugated nanoparticles using a surface acoustic wave sensor (SAW) comprises utilizing an electromagnetic property of the semiconductive structure to measure the number of DNA segments with conjugated nanoparticles.

5. The method of claim 4 where providing a semiconductive structure on or in the nanoparticle comprises providing a selectively doped semiconductive structure on or in the nanoparticle so that an active or passive electrical device is formed.

6. The method of claim 5 where providing a selectively doped semiconductive structure on or in the nanoparticle so that an active or passive electrical device comprises forming an antenna, diode or transistor on or in the semiconductive structure.

7. The method of claim 1 where conjugating a nanoparticle to the selected protein comprises conjugating a gold nanoparticle (GNP) prepared as a single electron transistor (SET) to the selected protein, and where measuring the number of DNA segments with conjugated nanoparticles using the surface acoustic wave sensor (SAW) comprises detecting an emitted SET event.

8. The method of claim 7 where detecting the emitted SET event comprises detecting an electrical output change as an output signal from the SET, thereby switching the dielectric gate and providing a measurable unit of detection.

9. A delivery system for use in a surface acoustic wave sensor (SAW) for isothermal detection of DNA comprising a plurality of delivery vehicles, each including (i) one or more guide RNA (gRNA) and (ii) a nucleic acid editing system, wherein the one or more gRNA is provided in a first delivery vehicle and the nucleic acid editing system is provided in a second delivery vehicle including a conjugated gold or iron nanoparticle, so that a limit of detection of the surface acoustic wave sensor (SAW) is improved.

10. The delivery system of claim 9 further comprising an active or passive selectively doped semiconductive provided in or on the gold nanoparticle, so that electromagnetic interaction with the semiconductive structure is utilized in the surface acoustic wave sensor (SAW) for detection.

11. A method comprising modifying a target nucleotide sequence in a DNA segment to enhance the mass of the DNA segment for detection by a surface acoustic wave sensor (SAW), the method comprising administering to the DNA segment a delivery system for isothermal detection of DNA comprising a plurality of delivery vehicles, each including (i) one or more guide RNA (gRNA) and (ii) a nucleic acid editing system, wherein the one or more gRNA is provided in a first delivery vehicle and the nucleic acid editing system is provided in a second delivery vehicle including a conjugated gold or iron nanoparticle, so that a limit of detection of the surface acoustic wave sensor (SAW) is improved.

12. An apparatus comprising:
a shear surface acoustic wave sensor (SAW) having a functionalized sensing area;
a plurality of at least one kind of selected target DNA segments used to functionalize the sensing area of the SAW; and
a plurality of gold-nanoparticle (GNP)-based single electron transistors (SETs) provided for selective conjugation with the plurality of at least one kind of selected target DNA segments using a CRISPR/Cas9 methodology, each SET having an RF responsive floating gate.

13. The apparatus of claim 12 in combination with an RF or ultrasonic source emitting an RF signal or ultrasonic wave signal respectively and where each of the gold-nanoparticle (GNP)-based single electron transistors (SETs) are chemically modified to function as a beacon to respond to the emitted RF signal and or the ultrasonic wave signal from the corresponding RF source or ultrasonic source by switching to the emitted RF energy or ultrasonic wave signal to generate a detectable emitted SET event.

14. The apparatus of claim 12 where the SET comprises a gold nanoparticle (GNP) formed as a transistor fabricated on a heavily doped n-type silicon substrate with a thermally grown oxide layer formed thereon of up to 100 nm thick, where the heavily doped n-type silicon substrate serves as a gate electrode of the transistor while the thermally grown oxide layer serves as a gate dielectric of the transistor.

15. The apparatus of claim 12 where a surface of the gold nanoparticle is configured as a single electron transistor-gate (SET) is provided with source/drain electrodes and where the gold nanoparticle is combined with a chemical or biochemical probe, and where detectable switching of the SET in response to an RF signal is indicative of binding of the target analyte to the probe.

16. The apparatus of claim 15 where the surface of the gold nanoparticle is configured as a single electron transistor-gate (SET) by chemical modification with linker molecules including guided RNA (gRNA) and where a channel region of the SET includes a linker layer composed of a self-assembled monomolecular layer formed by a plurality of organic mono-molecules bonded to the gold nanoparticle and a linker layer at least a portion of which is composed of a silane compound layer formed on the gold nanoparticle, the silane compound layer including a functional group selected from a class consisting of an amine group (—NH₂), a carboxyl group (—COOH) and a thiol group (—SH).

17. A method comprising:
functionalizing sensing area of a shear surface acoustic wave sensor (SAW) with a plurality of at least one kind of selected target DNA segments; and
providing a plurality of gold-nanoparticle (GNP)-based single electron transistors (SETs) provided for selective conjugation with the plurality of at least one kind of selected target DNA segments using a CRISPR/Cas9 methodology, each SET having an RF responsive floating gate

18. The method of claim 17 further comprising
providing an RF or ultrasonic source emitting an RF signal or ultrasonic wave signal respectively and where providing a plurality of gold-nanoparticle (GNP)-based single electron transistors (SETs) provides each of the gold-nanoparticle (GNP)-based single electron transistors (SETs) in a configuration which is chemically modified to function as a beacon to respond to the emitted RF signal and or the ultrasonic wave signal from the corresponding RF source or ultrasonic source;
switching at least some of the plurality of gold-nanoparticle (GNP)-based single electron transistors (SETs) to the emitted RF energy or ultrasonic wave signal; and
generating a detectable emitted SET event output.

19. The method of claim 17 where providing a plurality of gold-nanoparticle (GNP)-based single electron transistors (SETs) provides a gold nanoparticle (GNP) formed as a transistor fabricated on a heavily doped n-type silicon substrate with a thermally grown oxide layer formed thereon of up to 100 nm thick, where the heavily doped n-type silicon substrate serves as a gate electrode of the transistor while the thermally grown oxide layer serves as a gate dielectric of the transistor.

20. The method of claim 17 where providing a plurality of gold-nanoparticle (GNP)-based single electron transistors (SETs) provides a gold nanoparticle with a surface configured as a single electron transistor-gate (SET) and provided with source/drain electrodes and where the gold nanoparticle is combined with a chemical or biochemical probe, and where detectable switching of the SET in response to an RF signal is indicative of binding of the target analyte to the probe.

21. The method of claim 20 where a gold nanoparticle with a surface configured as a single electron transistor-gate (SET) comprising chemically modifying the surface with linker molecules including guided RNA (gRNA) and forming a channel region of the SET including a linker layer composed of a self-assembled monomolecular layer formed by a plurality of organic mono-molecules bonded to the gold nanoparticle and a linker layer at least a portion of which is composed of a silane compound layer formed on the gold nanoparticle, the silane compound layer including a functional group selected from a class consisting of an amine group (—NH₂), a carboxyl group (—COOH) and a thiol group (—SH).

22. The apparatus of claim 12 further comprising:
an array of a plurality of the shear surface acoustic wave sensors (SAW) each having a functionalized sensing area, each having a plurality of at least one kind of selected target DNA segments used to functionalize the sensing area of the SAW, and and each having a plurality of gold-nanoparticle (GNP)-based single electron transistors (SETs) provided for selective conjugation with the plurality of at least one kind of selected target DNA segments using a CRISPR/Cas9 methodology, each SET having an RF responsive floating gate; and
a SAW reader communicated with the array for detecting an output signal from each one of the plurality of shear surface acoustic wave sensors (SAW) and for generating a multiplexed corresponding digital data readout thereby allowing for simultaneous measurement of multiply different analytes.

23. The apparatus of claim 22 further comprising a source follower amplifier coupled with each shear surface acoustic wave sensors (SAW) as an output interface.

24. The apparatus of claim 12 where the shear surface acoustic wave sensor (SAW) is configured with a predetermined minimal mass loading above the SNR measure, where the shear surface acoustic wave sensor (SAW) has a sensing surface functionalized using a conjugation method of isothermal DNA editing employing CRISPR-Cas9 with an added mass of a GNP conjugated particle included in each of the gold-nanoparticle (GNP)-based single electron transistors to reduce the LOD, where each one of the plurality of gold-nanoparticle (GNP)-based single electron transistors (SETs) includes the GNP conjugated particle used as a marker detectable by use of an electro-acoustic wave and
further comprises:
a down conversion circuit communicated shear surface acoustic wave sensor (SAW) to enable a time domain suitable for signal capture of biological kinetics; and
an integrated microfluidic platform system to control hydrostatic flow and capture-rate of hybridization statistics.

25. The apparatus of claim 22 where the SAW reader comprises:
an RF source coupled to the array of a plurality of the shear surface acoustic wave sensors (SAW), each shear surface acoustic wave sensors (SAW) having an output;
an analog front end circuit for frequency down conversion having an input coupled to the outputs of the array;
an analog-to-digital converter, the analog front end circuit having outputs coupled the analog-to-digital converter; and
a digital I/Q demodulator, the analog-to-digital converter having an output coupled to the digital I/Q demodulator
whereby measurement is achieved with processing and analysis of amplitude or phase data in a time domain-scale at a rate of microseconds.
